(19) United States
(12) Patent Application Publication
(10) Pub. No.: US 2019/0282637 A1
(43) Pub. Date: Sep. 19, 2019

(54) COMPOSITIONS AND METHODS FOR IMPROVED RESTORATION AND PRESERVATION OF THE INTEGRITY OF TISSUE BARRIERS

(71) Applicant: Rejuvenation Science, Inc., Redondo Beach, CA (US)

(72) Inventor: Howard M. SIMON, Redondo Beach, CA (US)

(73) Assignee: Rejuvenation Science, Inc., Redondo Beach, CA (US)

(21) Appl. No.: 16/095,283

(22) PCT Filed: Apr. 19, 2017
(86) PCT No.: PCT/US2017/028416

§ 371 Date: Oct. 19, 2018

Related U.S. Application Data
(60) Provisional application No. 62/324,780, filed on Apr. 19, 2016.

(51) Int. Cl. A61K 35/747; A61K 35/20; A61K 35/744; A61K 35/745; A61K 35/38; A61K 45/06; A61K 35/742

(52) U.S. Cl. CPC A61K 35/747; A61K 35/20; A61K 35/744; A61K 35/742; A61K 35/38; A61K 45/06; A61K 35/745

(57) ABSTRACT

The present invention provides compositions (for example, nutritional compositions or therapeutic compositions) that comprise a probiotic component, colostrum, a protein component, and a detoxification component. The compositions may further comprise a prebiotic component, a tissue constituent component, a symptomatic relief component, a cellular bioenergetics component, a tissue healing component, and/or an enzyme component. Also provided are methods of preventing or treating a disturbance in the integrity of a tissue barrier and/or a mucosal membrane adjacent to or on a tissue of an individual and preventing or treating resulting diseases or conditions by administering such compositions to the individual.

### CROSS REFERENCE TO RELATED APPLICATIONS

This application claims priority to U.S. Provisional Patent Application No. 62/324,780, filed Apr. 19, 2016, the disclosure of which is incorporated herein by reference in its entirety.

### TECHNICAL FIELD

The present invention relates to compositions and methods for restoring and preserving the integrity of tissue barriers, such as the barriers in the gastrointestinal (“GI”) tract, sinuses, oral cavity, liver, kidneys, urinary tract, esophagus, vagina, lungs, cardiovascular system, lymphatic system, nervous system, and skin, and the blood/brain barrier, including uses thereof for preventing and/or treating diseases.

### BACKGROUND

Dysbiosis, an imbalance of the microorganisms living on or inside the body, has been associated with a number of serious illnesses, including inflammatory bowel disease (IBD), autoimmune and allergic diseases, cardiovascular diseases, and neurological diseases, to name a few. Dysbiosis may have diverse etiologies including a combination of antibiotic exposure, alcohol exposure, inappropriate diet, and exposure to environmental contaminants. Correcting dysbiosis and preserving a healthy balance of microorganisms may provide a therapeutic approach for preventing and treating a wide variety of disorders.

Gastrointestinal diseases such as IBD and irritable bowel syndrome (IBS) are common diseases in the U.S. Both IBS and IBD share symptoms of altered bowel habits associated with abdominal pain or discomfort. The most common symptoms of IBD and IBS include abdominal pain, cramping, bloody stools, diarrhea, fever, chills and dehydration. IBS refers to a functional bowel disorder, which is diagnosed by a characteristic cluster of symptoms in the absence of detectable structural abnormalities. IBD is a heterogeneous group of disorders characterized by various forms of chronic mucosal and/or transmural inflammation of the intestine.

Colitis is a one form of IBD. Most forms of colitis are mild to moderate, although an extreme condition known as ulcerative colitis can become debilitating over time, caused by the development of ulcers inside the colon or rectum. This condition is both chronic and progressive—it will get worse over time if not treated properly and could eventually manifest as Crohn's Disease. Crohn's Disease is one of the most severe forms of IBD.

Immune dysfunction, lack of blood supply, heredity, stress, and diet have all been proposed as potential causes of IBS or IBD. However, there is little information about the underlying causes of these diseases, and effective therapies are needed to prevent and treat the causes of these diseases, rather than their symptoms.

Autoimmune diseases are currently viewed as arising from an abnormal immune response of the body against substances and tissues normally present in the body (autoimmunity). This may be restricted to certain organs (e.g. in autoimmune thyroiditis) or involve a particular tissue in different places (e.g. Goodpasture's disease which may affect the basement membrane in both the lung and the kidney).

It has been estimated that autoimmune diseases are among the top ten leading causes of death among men and women in all age groups. A substantial minority of the population suffers from these diseases, which are often chronic, debilitating, and life-threatening. There are more than 80 illnesses categorized as autoimmune disease.

Western medicine has struggled to find effective treatments for autoimmune diseases. No cure or simple, universally effective treatment has been found yet for Grave's disease, lupus, or multiple sclerosis. The treatment of autoimmune diseases is typically with medication that treats symptoms and decreases the patient's entire immune response.

Cardiovascular disease refers to any disease that affects the cardiovascular system, principally cardiac disease, vascular diseases of the brain and kidney, and peripheral arterial disease. The causes of cardiovascular disease are diverse but arteriosclerosis, hypertension, and inflammation are the most commonly cited. In addition, with aging come a number of physiological and morphological changes that alter cardiovascular function and lead to increased risk of cardiovascular disease, even in healthy asymptomatic individuals.

Cardiovascular disease is the leading cause of deaths worldwide, though, since the 1970s, cardiovascular mortality rates have declined in many high-income countries. At the same time, cardiovascular deaths and disease have increased at a fast rate in low- and middle-income countries. Although cardiovascular disease usually affects older adults, the commonly accepted antecedents of cardiovascular disease, notably arteriosclerosis, begin in early life, making primary prevention efforts necessary from childhood. There is therefore increased emphasis on preventing arteriosclerosis by modifying risk factors, for example by healthy eating, exercise, and avoidance of smoking tobacco.

Without an understanding of the underlying cause or etiology of these diseases and the various pathways involved, conventional medicine has been hard pressed to find treatments other than for some of the symptoms of these diseases. The compositions and methods of the present invention provide new and useful compositions and methods for preventing, treating and/or intervening in the aforementioned diseases, as well as many other diseases and conditions as described below.

The disclosures of all publications, patents, patent applications and published patent applications referred to herein are hereby incorporated herein by reference in their entirety.

### BRIEF SUMMARY OF THE INVENTION

The present invention, in some embodiments, provides a composition (such as an oral composition) comprising: 1) a probiotic component, 2) colostrum, 3) a protein component and 4) a detoxification component comprising an ingredient that promotes toxin removal and/or free radical quenching. In some embodiments, the composition further comprises one or more components selected from the group consisting of 5) a prebiotic component, 6) a tissue constituent component comprising an organic constituent of a tissue, 7) a symptomatic relief component comprising an ingredient that provides symptomatic relief, 8) a cellular bioenergetics component comprising a nutrient that promotes cellular bioenergetics, 9) a tissue healing component comprising a nutrient that promotes healing of a tissue, and 10) an enzyme component comprising an enzyme that dissolves or reduces scabs, polyps, callouses and/or scars.

In some embodiments, the probiotic component comprises: 1) at least one microorganism of the Bacillus genus; 2) at least one microorganism of the Lactobacillus genus; 3) at least one microorganism of the Bifidobacterium genus, 4) at least one microorganism of the Escherichia genus, 5) at least one microorganism of the Streptococcus genus and 6) at least one microorganism of the Lactococcus genus. In some embodiments, the at least one microorganism of the Bacillus genus is one or more of: Bacillus coagulans and Bacillus subtilis. In some embodiments, the at least one microorganism of the Lactobacillus genus is one or more of: Lactobacillus paracaei, Lactobacillus casei, Lactobacillus bulgaricus, Lactobacillus acidophilus, Lactobacillus rhamnosis, Lactobacillus rhamnosis GR-1®, Lactobacillus plantarum, Lactobacillus johnsonii, Lactobacillus salivarius, Lactobacillus fermentum, Lactobacillus helveticus, Lactobacillus gasseri, Lactobacillus reuteri, Lactobacillus reuteri Protectis, Lactobacillus reuteri Prodentis and Lactobacillus reuteri RC-14®. In some embodiments, the at least one microorganism of the Bifidobacterium genus is one or more of: Bifidobacterium lactis, Bifidobacterium breve, Bifidobacterium longum, Bifidobacterium infantis, Bifidobacterium animalis subsp. lactis and Bifidobacterium bifidum. In some embodiments, the at least one microorganism of the Escherichia genus is E. coli. In some embodiments, the at least one microorganism of the Streptococcus genus is Streptococcus thermophiles. In some embodiments, the at least one microorganism of the Lactococcus genus is one or more of: Lactococcus thermophiles and Lactococcus lactis. In some embodiments, the probiotic component further comprises one or more of: L. cellobiosus, L. leichmannii and L. salivaroes. In some embodiments, the probiotic component comprises all the microorganisms of Table 1. In some embodiments, the probiotic component comprises all the microorganisms of Table 2.

In some embodiments, the colostrum is substantially non-allergenic. In some embodiments, the colostrum is from human, cow, sheep or goat. In some embodiments, the prebiotic component comprises one or more of: inulin, galactooligosaccharide (GOS), fructooligosaccharide (FOS), xylooligosaccharide (XOS), mannan-oligosaccharide (MOS) and polydextrose. In some embodiments, the prebiotic component comprises fructooligosaccharide.

In some embodiments, the probiotic component, colostrum, prebiotic component and protein component are provided in one of the amounts listed in Table 3. In some embodiments, the detoxification component comprises modified citrus pectin or one or more of the items listed in Table 4 for oral chelation and antioxidant detox formula. In some embodiments, the detoxification component comprises modified citrus pectin or one or more of the items listed in Table 4 for oral chelation and antioxidant detox formula in one of the amounts listed. In some embodiments, the tissue constituent component comprises one or more of the items listed in Table 5. In some embodiments, the tissue constituent component comprises one or more of the items listed in Table 5 in one of the amounts listed. In some embodiments, the organic constituent of a tissue is an organic constituent of the GI tissue. In some embodiments, the symptomatic relief component comprises one or more of the items listed in Table 6. In some embodiments, the symptomatic relief component comprises one or more of the items listed in Table 6 in one of the amounts listed. In some embodiments, the cellular bioenergetics component comprises one or more of the items listed in Table 7. In some embodiments, the cellular bioenergetics component comprises one or more of the items listed in Table 7 in one of the amounts listed. In some embodiments, the tissue healing component comprises one or more of the items listed in Table 8. In some embodiments, the tissue healing component comprises one or more of the items listed in Table 8 in one of the amounts listed. In some embodiments, the enzyme component comprises one or more of the items listed in Table 9. In some embodiments, the enzyme component comprises one or more of the items listed in Table 9 in one of the amounts listed.

Also provided is a method of enhancing, protecting or repairing the integrity of the tissue barrier adjacent to or on a tissue of an individual, comprising administering to the individual an effective amount of any of the compositions described herein. In some embodiments, there is provided a method of preventing or treating inflammation of a tissue of an individual, comprising administering to the individual an effective amount of any of the compositions described herein. In some embodiments, there is provided a method of preventing or repairing damage in an individual, comprising administering to the individual an effective amount of any of the compositions described herein. In some embodiments, the damage is tissue damage. In some embodiments, there is provided a method of mitigating leakage of a tissue barrier and/or mucosal membrane adjacent to or on a tissue of an individual, comprising administering to the individual an effective amount of any of the compositions described herein. In some embodiments, there is provided a method of reducing the risk of a disease or condition in an individual, comprising administering to the individual an effective amount of any of the compositions described herein. In some embodiments, there is provided a method of reducing a side effect associated with the administration of an antibiotic in an individual, comprising administering to the individual an effective amount of any of the compositions described herein. In some embodiments, there is provided a method of reducing an adverse effect associated with the exposure of an individual to an environmental contaminant or non-natural substance, comprising administering to the individual an effective amount of any of the compositions described herein.

In some embodiments, there is provided a method of preventing or treating a disease or condition in an individual, comprising administering to the individual an effective amount of any of the compositions described herein. In some embodiments, there is provided a method of preventing or treating a disease or condition in an individual, comprising administering to the individual an effective amount of a plurality of compositions comprising 1) a probiotic component, 2) colostrum, 3) a protein component and 4) a detoxification component. In some embodiments, the plurality of compositions further comprises one or more of 5) a prebiotic component, 6) a tissue constituent component, 7) a symptomatic relief component, 8) a cellular bioenergetics component, 9) a tissue healing component, and 10) an enzyme component. In some embodiments, the disease or condition is associated with a disturbance in the integrity of one or more tissue barriers adjacent to or on one or more tissues in the individual. In some embodiments, the disease or condition is selected from the group consisting of GI conditions, respiratory conditions, skin conditions, and autoimmune diseases. In some embodiments, the method further comprises determining a manifestation stage of the disease or condition and selecting the composition or plurality of compositions according to the affected tissues and manifestation stage.

In some embodiments, there is provided a method of simultaneously treating comorbid conditions in an individual, comprising administering to the individual an effective amount of any of the compositions described herein. In some embodiments, there is provided a method of simultaneously enhancing, protecting or repairing the integrity of multiple tissue barriers located in different regions in the body of an individual, comprising administering to the individual an effective amount of any of the compositions described herein. In some embodiments, there is provided a method of enhancing, protecting or repairing the integrity of the blood-brain barrier, comprising administering to the individual an effective amount of any of the compositions described herein. In some embodiments, there is provided a method of treating diseases associated with brain or neurological dysfunction, comprising administering to the individual an effective amount of any of the compositions described herein. In some embodiments, there is provided a method of reducing the risk of one or more diseases associated with inflammation, comprising administering to the individual an effective amount of any of the compositions described herein. In some embodiments, the diseases associated with inflammation are selected from the group consisting cardiovascular disease, food allergies, and sinus allergies. In some embodiments, there is provided a method of treating diseases associated with exposure to one or more contaminants, comprising administering to the individual an effective amount of any of the compositions described herein. In some embodiments, the contaminants are selected from the group consisting of chlorine, herbicides, pesticides, antibiotics in foods and drinking or bathing water, hydrocarbons, tar, nicotine, smoke, and heavy metals. In some embodiments, there is provided a method of protecting a tissue after surgery and promoting post-surgical healing of the tissue, comprising administering to the individual an effective amount of any of the compositions described herein. In some embodiments, the tissue is GI tissue and the surgery is GI surgery.

In some embodiments of any of the methods described herein, the individual is an infant, a child, an adult or a pregnant female. In some embodiments, the individual is human.

The present application also provides kits and unit doses for the compositions described herein.

### DETAILED DESCRIPTION OF THE INVENTION

The present invention in one aspect provides a composition (such as a nutritional composition or a pharmaceutical composition) that restores and/or preserves the integrity of one or more tissue barriers (e.g., intestinal milieu) adjacent to or on one or more tissues in an individual. The present application is based on the inventor's insight that a composition comprising a probiotic component, colostrum, a protein component and a detoxification component has therapeutic and preventive effects against various diseases and conditions, such as autoimmune diseases and diseases of the gastrointestinal tract, due to its beneficial properties in preserving the integrity of tissue barriers and preventing their leakage.

The present application in one aspect provides a composition comprising a probiotic component, colostrum, a protein component and a detoxification component. In another aspect, the composition further comprises one or more components selected from the group consisting of a prebiotic component, a tissue constituent component, a symptomatic relief component, a cellular bioenergetics component, a tissue healing component, and an enzyme component.

In yet another aspect, there are provided methods of using a composition or plurality of compositions as described herein for various purposes, which include, but are not limited to, enhancing, protecting or repairing the integrity of the tissue barrier adjacent to or on a tissue, preventing or treating inflammation of a tissue, preventing or treating diseases and conditions associated with disturbance of one or more tissue barriers adjacent to or on one or more tissues and alleviating symptoms associated with disturbance of a tissue barrier adjacent to or on a tissue.

In another aspect, there are provided methods of mitigating leakage of a tissue barrier and/or a mucosal membrane adjacent to or on a tissue, reducing the risk of a disease or condition associated with a disturbance in the integrity of one or more tissue barriers adjacent to or on one or more tissues of an individual, reducing a side effect associated with the administration of an antibiotic, and reducing an adverse effect associated with the exposure to an environmental contaminant or non-natural substance, by administering an effective amount of a composition or plurality of compositions as described herein.

In some embodiments, the composition is provided as an oral composition, a topical composition, a sinus mist, an enema or suppository, drops, a formulation suitable for parenteral administration, or a combination thereof depending on the dosage form requirements for the ingredients indicated for the tissue and/or condition being treated

Also provided are unit dosages, article of manufacture, and kits comprising the compositions described herein useful for the methods described herein.

### Definitions

A “probiotic component” used herein refers to a combination of beneficial microorganisms.

A “prebiotic component” used herein refers to one or more food source for probiotics.

“Colostrum” used herein refers to a form of milk produced by the mammary glands of mammals in late pregnancy and for a short period after birth. It is typically rich in carbohydrates, protein and antibodies.

A “tissue barrier” used herein refers to a structure lining a tissue comprising host cells, proteins, and any associated microorganisms, and is also meant to encompass the surrounding microenvironment. Tissue barriers thus include, but are not limited to, the intestinal milieu, the gastric milieu, the sinus milieu, the esophageal milieu, the vascular milieu, the pulmonary milieu, the dermal or epithelial milieu, the blood-brain barrier, the hepatic milieu, the renal milieu, the vaginal milieu, the testicular milieu, the urethral milieu, the oral milieu and the periodontal milieu.

The amount of a given prebiotic or probiotic in the composition includes both those inherently present in the colostrum and those added as part of the prebiotic or probiotic component. For example, a composition “comprises X amount of a prebiotic” means that the total amount of the probiotic in the composition is X. A probiotic component “comprises X amount of a probiotic” means that the total amount of the probiotic added in addition to those inherently present in the colostrum is X.

All percentages and ratios as used herein unless otherwise specified are by weight of the total composition. All such weights as they pertain to listed ingredients are based on the active level and therefore do not include the weight of carriers or impurities that may be included in the commercially available materials, unless otherwise specified. All percentages and ratios related to microorganisms and probiotics are by the number of colony forming units (CFU).

Reference to “about” a value or parameter herein includes (and describes) embodiments that are directed to that value or parameter per se. For example, description referring to “about X” includes description of “X.”

The compositions and methods of the present invention may be substantially free of a specific ingredient described herein. In this context, the term “substantially free” means that the compositions comprise less than about 2%, including less than about 0.5%, less than about 0.1%, or 0%, by weight of the specific ingredient.

The compositions and methods of the present invention may comprise, consist of, or consist essentially of the essential elements and limitations of the invention described herein, as well as any additional or optional ingredients, components, or limitations described herein or otherwise useful in a nutritional or pharmaceutical application.

### Compositions of the Present Invention

The present invention in some embodiments provides a composition (such as an oral composition) comprising a probiotic component, colostrum, a protein component and a detoxification component. In some embodiments, the composition further comprises one or more components selected from the group consisting of a prebiotic component, a tissue constituent component, a symptomatic relief component, a cellular bioenergetics component, a tissue healing component, and an enzyme component. The compositions are further described below in more detail.

In some embodiments, the probiotic component comprises at least one (such as at least any of 2, 3, 4, 5, 6, 7, 8, 9, or 10) microorganism of the following: a microorganism of the Bacillus genus, a microorganism of the Lactobacillus genus, a microorganism of the Bifidobacterium genus, a microorganism of the Escherichia genus, a microorganism of the Streptococcus genus, and a microorganism of the Lactococcus genus. In some embodiments, the probiotic component comprises at least one (such as at least any of 2, 3, 4, 5, 6, 7, 8, 9, or 10) microorganism of the Bacillus genus. In some embodiments, the probiotic component comprises at least one (such as at least any of 2, 3, 4, 5, 6, 7, 8, 9, or 10) microorganism of the Lactobacillus genus. In some embodiments, the probiotic component comprises at least one (such as at least any of 2, 3, 4, 5, 6, 7, 8, 9, or 10) microorganism of the Bifidobacterium genus. In some embodiments, the probiotic component comprises at least one (such as at least any of 2, 3, 4, 5, 6, 7, 8, 9, or 10) microorganism of the Escherichia genus. In some embodiments, the probiotic component comprises at least one (such as at least any of 2, 3, 4, 5, 6, 7, 8, 9, or 10) microorganism of the Streptococcus genus. In some embodiments, the probiotic component comprises at least one (such as at least any of 2, 3, 4, 5, 6, 7, 8, 9, or 10) microorganism of the Lactococcus genus. In some embodiments, the probiotic component further comprises one or more of: L. cellobiosus, L. leichmannii and L. salivaroes. In some embodiments, the probiotic component further comprises Saccharomyces boulardii.

In some embodiments, the probiotic component comprises at least one (such as at least any of 2, 3, 4, 5, 6, 7, 8, 9, or 10) microorganism of the Bacillus genus, and at least one (such as at least 2) of the microorganisms is selected from the group consisting of: Bacillus coagulans and Bacillus subtilis.

In some embodiments, the probiotic component comprises at least one (such as at least any of 2, 3, 4, 5, 6, 7, 8, 9, or 10) microorganism of the Lactobacillus genus, and at least one (such as at least 2, 3, 4, 5, 6, 7, 8, 9, 10, 11, 12, 13, 14, 15, 16 or 17) of the microorganisms is selected from the group consisting of: Lactobacillus paracaei, Lactobacillus casei, Lactobacillus bulgaricus, Lactobacillus acidophilus, Lactobacillus rhamnosis, Lactobacillus rhamnosis GR-1®, Lactobacillus plantarum, Lactobacillus johnsonii, Lactobacillus salivarius, Lactobacillus fermentum, Lactobacillus helveticus, Lactobacillus gasseri, Lactobacillus reuteri, Lactobacillus reuteri Protectis, Lactobacillus reuteri Prodentis, Lactobacillus reuteri RC-14® and Lactobacillus brevis.

In some embodiments, the probiotic component comprises at least one (such as at least any of 2, 3, 4, 5, 6, 7, 8, 9, or 10) microorganism of the Bifidobacterium genus, and at least one (such as at least 2, 3, 4, 5, or 6) of the microorganisms is selected from the group consisting of: Bifidobacterium lactis, Bifidobacterium breve, Bifidobacterium longum, Bifidobacterium infantis, Bifidobacterium animalis subsp. lactis, and Bifidobacterium bifidum.

In some embodiments, the probiotic component comprises at least one (such as at least any of 2, 3, 4, 5, 6, 7, 8, 9, or 10) microorganism of the Escherichia genus, and one of the microorganisms is E. coli.

In some embodiments, the probiotic component comprises at least one (such as at least any of 2, 3, 4, 5, 6, 7, 8, 9, or 10) microorganism of the Streptococcus genus, and one of the microorganisms is Streptococcus thermophiles.

In some embodiments, the probiotic component comprises at least one (such as at least any of 2, 3, 4, 5, 6, 7, 8, 9, or 10) microorganism of the Lactococcus genus, and at least one (such as at least 2) of the microorganisms is selected from the group consisting of: Lactococcus thermophiles and Lactococcus lactis.

In some embodiments, the probiotic component comprises at least one (such as at least any of 2, 3, 4, 5 or 6) microorganism selected from the group consisting of: a microorganism that promotes integrity of a tissue barrier, a microorganism that promotes intra-bacterial signaling, a microorganism that promotes healthy extra-bacterial signaling or signaling between a tissue barrier and a tissue, a microorganism that thrives in the pH range of a tissue barrier surrounding a damaged tissue (such as GI tissue), a microorganism that has high affinity for a tissue, and a microorganism that is commensal to one or more microorganisms present in a tissue barrier. In some embodiments, the probiotic component comprises at least one (such as at least any of 2, 3, 4, 5. 6, 7, 8, 9, or 10) microorganism that promotes integrity of the bacterial milieu. In some embodiments, the probiotic component comprises at least one (such as at least any of 2, 3, 4, 5, 6, 7, 8, 9, or 10) microorganism that promotes intra-bacterial signaling. In some embodiments, the probiotic component comprises at least one (such as at least any of 2, 3, 4, 5, 6, 7, 8, 9, or 10) microorganism that promotes healthy extra-bacterial signaling or signaling between the bacterial milieu and tissue. In some embodiments, the probiotic component comprises at least one (such as at least any of 2, 3, 4, 5, 6, 7, 8, 9, or 10) microorganism that thrives in the pH range of the tissue barrier surrounding a damaged tissue (such as a GI tissue). In some embodiments, the probiotic component comprises at least one (such as at least any of 2, 3, 4, 5, 6, 7, 8, 9, or 10) microorganism that has high affinity for a tissue. In some embodiments, the probiotic component comprises at least one (such as at least any of 2, 3, 4, 5, 6, 7, 8, 9, or 10) microorganism that is commensal to one or more microorganisms present in a tissue barrier.

In some embodiments, the probiotic component comprises at least one (such as at least any of 2, 3, 4, 5, 6, 7, 8, 9, or 10) microorganism selected from the group consisting of: a microorganism that adheres to the upper portion of the GI tract, a microorganism that adheres to the middle portion of the GI tract, a microorganism that adheres to the bottom portion of the GI tract, a microorganism that adheres to oral/periodontal tissue, a microorganism that adheres to vascular tissue, a microorganism that adheres to hepatic tissue, a microorganism that adheres to renal tissue, a microorganism that adheres to dermal tissue, a microorganism that adheres to the urinary tract, a microorganism that adheres to sinus tissue, a microorganism that adheres to esophageal tissue, a microorganism that adheres to pulmonary tissue, and a microorganism that adheres to vaginal tissue. In some embodiments, the probiotic component comprises at least one (such as at least any of 2, 3, 4, 5. 6, 7, 8, 9, or 10) microorganism that adheres to the upper portion of the GI tract. In some embodiments, the probiotic component comprises at least one (such as at least any of 2, 3, 4, 5, 6, 7, 8, 9, or 10) microorganism that adheres to the middle portion of the GI tract. In some embodiments, the probiotic component comprises at least one (such as at least any of 2, 3, 4, 5, 6, 7, 8, 9, or 10) microorganism that adheres to the bottom portion of the GI tract. In some embodiments, the probiotic component comprises at least one (such as at least any of 2, 3, 4, 5, 6, 7, 8, 9, or 10) microorganism that adheres to oral/periodontal tissue. In some embodiments, the probiotic component comprises at least one (such as at least any of 2, 3, 4, 5, 6, 7, 8, 9, or 10) microorganism that adheres to vascular tissue. In some embodiments, the probiotic component comprises at least one (such as at least any of 2, 3, 4, 5. 6, 7, 8, 9, or 10) microorganism that adheres to hepatic tissue. In some embodiments, the probiotic component comprises at least one (such as at least any of 2, 3, 4, 5. 6, 7, 8, 9, or 10) microorganism that adheres to renal tissue. In some embodiments, the probiotic component comprises at least one (such as at least any of 2, 3, 4, 5. 6, 7, 8, 9, or 10) microorganism that adheres to dermal tissue. In some embodiments, the probiotic component comprises at least one (such as at least any of 2, 3, 4, 5. 6, 7, 8, 9, or 10) microorganism that adheres to the urinary tract. In some embodiments, the probiotic component comprises at least one (such as at least any of 2, 3, 4, 5. 6, 7, 8, 9, or 10) microorganism that adheres to sinus tissue. In some embodiments, the probiotic component comprises at least one (such as at least any of 2, 3, 4, 5. 6, 7, 8, 9, or 10) microorganism that adheres to esophageal tissue. In some embodiments, the probiotic component comprises at least one (such as at least any of 2, 3, 4, 5. 6, 7, 8, 9, or 10) microorganism that adheres to pulmonary tissue. In some embodiments, the probiotic component comprises at least one (such as at least any of 2, 3, 4, 5. 6, 7, 8, 9, or 10) microorganism that adheres to vaginal tissue.

In some embodiments, the probiotic component comprises at least one (such as at least any of 2, 3, 4, 5, 6, 7, 8, 9, or 10) microorganism listed in Table 1. In some embodiments, the probiotic component comprises all the microorganisms listed in Table 1. Thus, in some embodiments, the probiotic component comprises Lactobacillus acidophilus (La-14), Bifidobacterium lactis (Bl-04), Lactobacillus plantarum (Lp-115), Bifidobacterium longum (Bl-05), Lactobacillus rhamnosus (Lr-32), Streptococcus thermophiles (St-21), Lactobacillus paracasei (Lpc-37), Bifidobacterium breve (Bb-03), Lactobacillus salivarious (Ls-33) and Lactobacillus casei (Lc-11). In some embodiments, the probiotic component comprises, in order of highest to lowest weight contribution, Lactobacillus acidophilus (La-14), Bifidobacterium lactis (Bl-04), Lactobacillus plantarum (Lp-115), Bifidobacterium longum (Bl-05), Lactobacillus rhamnosus (Lr-32), Streptococcus thermophiles (St-21), Lactobacillus paracasei (Lpc-37), Bifidobacterium breve (Bb-03), Lactobacillus salivarious (Ls-33) and Lactobacillus casei (Lc-11). In some embodiments, the probiotic component comprises, in order of highest to lowest CFU contribution, Lactobacillus acidophilus (La-14), Bifidobacterium lactis (Bl-04), Lactobacillus plantarum (Lp-115), Bifidobacterium longum (Bl-05), Lactobacillus rhamnosus (Lr-32), Streptococcus thermophiles (St-21), Lactobacillus paracasei (Lpc-37), Bifidobacterium breve (Bb-03), Lactobacillus salivarious (Ls-33) and Lactobacillus casei (Lc-11). In some embodiments, the probiotic component comprises Lactobacillus acidophilus (La-14), Bifidobacterium lactis (Bl-04), Lactobacillus plantarum (Lp-115), Bifidobacterium longum (Bl-OS), Lactobacillus rhamnosus (Lr-32), Streptococcus thermophiles (St-21), Lactobacillus paracasei (Lpc-37), Bifidobacterium breve (Bb-03), Lactobacillus salivarious (Ls-33) and Lactobacillus casei (Lc-11) at a weight ratio of about 21.7:21.7:20:1:1:1:1:1:1:1, respectively. In some embodiments, the probiotic component comprises Lactobacillus acidophilus (La-14), Bifidobacterium lactis (Bl-04), Lactobacillus plantarum (Lp-115), Bifidobacterium longum (Bl-05), Lactobacillus rhamnosus (Lr-32), Streptococcus thermophiles (St-21), Lactobacillus paracasei (Lpc-37), Bifidobacterium breve (Bb-03), Lactobacillus salivarious (Ls-33) and Lactobacillus casei (Lc-11) at a CFU ratio of about 21.7:21.7:20:1:1:1:1:1:1:1, respectively. In some embodiments, the probiotic component comprises Lactobacillus acidophilus (La-14), Bifidobacterium lactis (Bl-04), Lactobacillus plantarum (Lp-115), Bifidobacterium longum (Bl-05), Lactobacillus rhamnosus (Lr-32), Streptococcus thermophiles (St-21), Lactobacillus paracasei (Lpc-37), Bifidobacterium breve (Bb-03), Lactobacillus salivarious (Ls-33) and Lactobacillus casei (Lc-11), wherein the CFUs provided for each microorganism are within no more than about 10% (such as within no more than about any of 2%, 4%, 6%, 8% or 10%) of each other. In some embodiments, the total number of microorganism CFU in the probiotic component is provided according to Table 3. In some embodiments, the total number of microorganism CFU in the probiotic component is from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed in Table 3. Thus, in some embodiments, the probiotic component comprises a total of 30-60 billion microorganism CFU for a maintenance dose, 60-120 billion microorganism CFU for a stage 1 dose, 120-240 billion microorganism CFU for a stage 2 dose, 180-360 billion microorganism CFU for a stage 3 dose, 180-720 billion microorganism CFU for a stage 4 dose or 120-360 billion microorganism CFU for a leaky gut dose. In some embodiments, the probiotic component further comprises Saccharomyces boulardii (for example in an amount of about 10 billion CFU). Compositions comprising Saccharomyces boulardii are particularly suitable, for example, for individuals having diarrhea.

In some embodiments, the probiotic component comprises at least one (such as at least any of 2, 3, 4, 5, 6, 7, or 8) microorganism listed in Table 2. In some embodiments, the probiotic component comprises all the microorganisms listed in Table 2. Thus, in some embodiments, the probiotic component comprises Streptococcus thermophiles, Bifidobacterium breve, Bifidobacterium longum, Bifidobacterium infantis, Lactobacillus acidophilus, Lactobacillus plantarum, Lactobacillus paracasei and Lactobacillus delbrueckii subsp. Bulgaricus. In some embodiments, the probiotic component comprises from 30-35% by weight of lyophilized Streptococcus thermophiles, from 27-30% by weight of a mixture of lyophilized Bifidobacterium breve, Bifidobacterium longum and Bifidobacterium infantis, from 7-10% by weight of lyophilized Lactobacillus acidophilus, from 8-10% by weight of lyophilized Lactobacillus plantarum, from 7-10% by weight of lyophilized Lactobacillus paracasei and from 8-10% by weight of lyophilized Lactobacillus delbrueckii subsp. Bulgaricus. In some embodiments, the probiotic component comprises a) Streptococcus thermophiles, b) a mixture of Bifidobacterium breve, Bifidobacterium longum and Bifidobacterium infantis, c) Lactobacillus acidophilus, d) Lactobacillus plantarum, e) Lactobacillus paracasei and f) Lactobacillus delbrueckii subsp. Bulgaricus at a weight ratio of about 3.8:3.4:1:1:1:1, respectively. In some embodiments, the bifidobacteria mixture is about 1:1:1 by weight of Bifidobacterium breve, Bifidobacterium longum and Bifidobacterium infantis. In some embodiments, the probiotic component comprises Streptococcus thermophiles, Bifidobacterium breve, Bifidobacterium longum, Bifidobacterium infantis, Lactobacillus acidophilus, Lactobacillus plantarum, Lactobacillus paracasei and Lactobacillus delbrueckii subsp. Bulgaricus, wherein the CFUs provided for each microorganism are within no more than about 10% (such as within no more than about any of 2%, 4%, 6%, 8% or 10%) of each other. In some embodiments, the total number of microorganism CFU in the probiotic component is provided according to Table 3. In some embodiments, the total number of microorganism CFU in the probiotic component is from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed in Table 3. Thus, in some embodiments, the probiotic component comprises a total of 30-60 billion microorganism CFU for a maintenance dose, 60-120 billion microorganism CFU for a stage 1 dose, 120-240 billion microorganism CFU for a stage 2 dose, 180-360 billion microorganism CFU for a stage 3 dose, 180-720 billion microorganism CFU for a stage 4 dose or 120-360 billion microorganism CFU for a leaky gut dose. In some embodiments, the probiotic component further comprises Saccharomyces boulardii (for example in an amount of about 10 billion CFU). Compositions comprising Saccharomyces boulardii are particularly suitable, for example, for individuals having diarrhea.

In some embodiments, the probiotic component comprises microorganisms derived from human or animal sources, such as, but not limited to, feces, saliva, mucus, ear wax, blood, and tissue lining. In some embodiments, the probiotic component is derived from the lining of tissues including, but not limited to, the GI tract, oral cavity, and sinuses. The microorganisms derived from human or animal sources can be harvested using any techniques known in the art for the separation and preparation of microorganisms from a biological sample.

In some embodiments, when external factors render the probiotic component of the composition less effective, such as when used in conjunction with antibiotic therapy or when refrigeration of the composition is not possible, the probiotic component further comprises Bacillus coagulans at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed in Table 3.

In some embodiments, the microorganisms of the probiotic component are acid-resistant. In some embodiments, the probiotic component comprises greater than about 4 times the indicated number of CFUs of each of the microorganisms at production, e.g., 110 billion CFUs of a microorganism are provided at production where 25 billion CFUs are indicated. Thus, in some embodiments, the actual CFUs of a given microorganism of the probiotic component will vary between greater than about 4 times the indicated numbers at the time of production to about the indicated numbers at the time of expiration.

TABLE 1
Preferred probiotic formula 1

Lactobacillus acidophilus (La-14)
Bifidobacterium lactis (Bl-04)
Lactobacillus plantarum (Lp-115)
Bifidobacterium longum (Bl-05)
Lactobacillus rhamnosus (Lr-32)
Streptococcus thermophiles (St-21)
Lactobacillus paracasei (Lpc-37)
Bifidobacterium breve (Bb-03)
Lactobacillus salivarious (Ls-33)
Lactobacillus casei (Lc-11)

TABLE 2
Preferred probiotic formula 2 (VSL3 ®)

Streptococcus thermophiles
Bifidobacterium breve
Bifidobacterium longum
Bifidobacterium infantis
Lactobacillus acidophilus
Lactobacillus plantarum
Lactobacillus paracasei
Lactobacillus delbrueckii subsp. bulgaricus

TABLE 3
Typical daily dosages of components of the tissue barrier formula

| | Maintenance | | Stage 1 | | Stage 2 | | Stage 3 | | Stage 4 | | Leaky gut | |
|---|---|---|---|---|---|---|---|---|---|---|---|---|
| Component | Dose | | Dose | | Dose | | Dose | | Dose | | Dose | |
| Tissue barrier formula | | | | | | | | | | | | |
| Probiotic component¹ | 30-60B⁴ | | 60-120B | | 120-240B | | 180-360B | | 180-720B | | 120-360B | |
| Colostrum | 1-2 | g | 2-4 | g | 4-8 | g | 6-12 | g | 6-24 | g | 4-12 | g |
| Prebiotic component | 250-500 | mg | 0.5-1 | g | 1-2 | g | 1.5-3 | g | 1.5-6 | g | 1-3 | g |
| Whey protein | 5-10 | g | 10-20 | g | 20-40 | g | 30-60 | g | 30-120 | g | 20-60 | g |
| Beef protein² | 5-10 | g | 10-20 | g | 20-40 | g | 30-60 | g | 30-120 | g | 20-60 | g |

| | | | | | | |
|---|---|---|---|---|---|---|
| Bacillus coagulans³ | 5B | 10B | 20B | 20B | 20B | 20B |

¹Each strain may be present in equal numbers of CFUs, or weighted as described for preferred formulas 1 and 2.
²Optional, alternative to whey protein for pateints who are allergic/sensitive to milk/casein products.
³Optional, for use in conjunction with probiotic component when environmental factors make the preferred probiotic component less effective, such as with antibiotic therapy or when refrigeration is not possible.
⁴Billion colony forming units (CFU)

TABLE 4
Typical daily dosages of ingredients that promote
toxin removal and/or free radical quenching

| | | | Stage 1-4/ | |
|---|---|---|---|---|
| Microcontaminant/Free Radical | Maintenance | | Leaky Gut | |
| Treatment | Dose | | Dose | |
| Modified Citrus Pectin¹ | 5-10 | g | 10-20 | g |
| Oral Chelation and Antioxidant Detox | | | | |
| Formula^{1,2} | | | | |
| Calcium Disodium EDTA | 500 | mg | 500 | mg |
| N-Acetyl Cysteine | 500 | mg | 500 | mg |
| Chlorella | 375 | mg | 375 | mg |
| Rosemary Leaf Extract | 275 | mg | 275 | mg |
| Turmeric Root Extract (95% curcuminoids) | 250 | mg | 250 | mg |
| Modified Citrus Pectin | 250 | mg | 250 | mg |
| Garlic Bulb | 250 | mg | 250 | mg |
| Broccoli Sprouts (0.4% sulforaphane) | 250 | mg | 250 | mg |
| Holy Basil Leaf Extract | 225 | mg | 225 | mg |
| Olive Leaf Extract (10% oleuropein) | 166 | mg | 166 | mg |
| Silymarin | 150 | mg | 150 | mg |
| Calcium D-Glucarate | 125 | mg | 125 | mg |
| Green Tea Leaf Extract | 125 | mg | 125 | mg |
| Cilantro Leaf Extract | 125 | mg | 125 | mg |
| Wasabi Rhizome | 104 | mg | 104 | mg |
| Shilajit Mineral Resin Extract | 62 | mg | 62 | mg |
| R-Lipoic Acid | 50 | mg | 50 | mg |
| Coenzyme Q10 | 5 | mg | 5 | mg |
| Black Pepper Fruit Extract | 2.5 | mg | 2.5 | mg |
| Plasma chelation therapy^{1,3} | | | | |
| Antioxidants⁴ | | | | |

¹Alternative toxin treatments, only one used at a time.
²Suggested use for Oral Chelation and Antioxidant Detox Formula: 1st cycle - ⅓ dose twice daily for 5 days then 9 days off; 2nd cycle - ⅔ dose twice daily for 5 days then 9 days off; 3rd cycle - full dose twice daily for 5 days then 30 days off. Same dose and cycle for all conditions being treated. Take on an empty stomach with at least 12 ounces of water.
³Administered by a skilled professional.
⁴Free radical treatment, preferably recycling antioxidant blend such as Rejuvenation Science ® Green C ™, used in combination with any of the toxin treatments.

TABLE 5
Typical daily dosages of organic constituents of tissue

| | Maintenance | | Stage 1 | | Stage 2 | | Stage 3 | | Stage 4 | | Leaky gut | |
|---|---|---|---|---|---|---|---|---|---|---|---|---|
| Component | Dose | | Dose | | Dose | | Dose | | Dose | | Dose | |
| Lipid replacement: | | | | | | | | | | | | |
| Krill or green-lipped mussel oil | 0.5 | g | 0.5-1 | g | 1-2 | g | 2-4 | g | 4-8 | g | 1-3 | g |
| Lipid replacement¹: | | | | | | | | | | | | |
| Fish oil | 3 | g | 3-5 | g | 5-10 | g | 10-20 | g | 20-40 | g | 5-15 | g |
| Phospholipids | 150 | mg | 150-300 | mg | 300-600 | mg | 600-1200 | mg | 1.2-2.4 | g | 300-900 | mg |
| L-glutamine | 0.5 | g | 0.75 | g | 1.5 | g | 3 | g | 4.5 | g | 1.5-3 | g |
| N-acetyl glucosamine | 0.25 | g | 0.5 | g | 1 | g | 2 | g | 3 | g | 1-2 | g |
| Methyl-sulfonyl-methane | 25 | mg | 50 | mg | 100 | mg | 200 | mg | 300 | mg | 100-200 | mg |
| Vitamin D3² | | | | | | | | | | | | |
| Multivitamin³ | | | | | | | | | | | | |
| Zinc carnosine⁴ | 25 | mg | 50 | mg | 75 | mg | 150 | mg | 225 | mg | 75-150 | mg |
| Mucin⁴ | 25 | mg | 50 | mg | 100 | mg | 200 | mg | 300 | mg | 100-200 | mg |
| Sodium butyrate⁴ | 25 | mg | 50 | mg | 100 | mg | 200 | mg | 300 | mg | 100-200 | mg |
| Magnesium⁵ | 25 | mg | 50 | mg | 100 | mg | 200 | mg | 300 | mg | 100-200 | mg |
| Taurine⁵ | 25 | mg | 50 | mg | 100 | mg | 200 | mg | 300 | mg | 100-200 | mg |
| L-arginine⁵ | 0.5 | g | 0.75 | g | 1.5 | g | 3 | g | 4.5 | g | 1.5-3 | g |
| Silica⁶ | 25 | mg | 50 | mg | 100 | mg | 200 | mg | 300 | mg | 100-200 | mg |
| Phosphatidyl Serine⁷ | 25 | mg | 50 | mg | 100 | mg | 200 | mg | 300 | mg | 100-200 | mg |
| DMAE⁷ | 25 | mg | 50 | mg | 100 | mg | 200 | mg | 300 | mg | 100-200 | mg |
| Niacinamide⁷ | 25 | mg | 50 | mg | 100 | mg | 200 | mg | 300 | mg | 100-200 | mg |
| CMO⁸ | 25 | mg | 50 | mg | 100 | mg | 200 | mg | 300 | mg | 100-200 | mg |
| Hyaluronic acid⁸ | 25 | mg | 50 | mg | 100 | mg | 200 | mg | 300 | mg | 100-200 | mg |
| Glucosamine⁸ | 25 | mg | 50 | mg | 100 | mg | 200 | mg | 300 | mg | 100-200 | mg |
| Chondroitin⁸ | 25 | mg | 50 | mg | 100 | mg | 200 | mg | 300 | mg | 100-200 | mg |

¹Optional, alternative to lipid replacement with krill or green-lipped mussel oil.
²Dose is titrated to achieve 80-100 ng/ml 25-hydroxyvitamin D [25(OH)D] plasma level (typically 5,000 to 10,000 IU/day)
³Standard daily dose of complex multivitamin, such as Rejuvenation Science Maximum Vitality
⁴Indicated for GI conditions
⁵Indicated for cardiovascular conditions
⁶Indicated for skin conditions
⁷Indicated for brain conditions
⁸Indicated for joint conditions

TABLE 6
Typical daily dosages of ingredients providing symptomatic relief

| | Maintenance | | Stage 1 | | Stage 2 | | Stage 3 | | Stage 4 | | Leaky gut | |
|---|---|---|---|---|---|---|---|---|---|---|---|---|
| Component | Dose | | Dose | | Dose | | Dose | | Dose | | Dose | |
| Citrus pectin¹ | 0.25 | g | 0.5 | g | 1 | g | 2 | g | 3 | g | 1-2 | g |
| Deglycyrrhizinated licorice¹ | 125 | mg | 250 | mg | 400 | mg | 800 | mg | 1.2 | g | 400-800 | mg |
| Aloe vera extract¹ | 100 | mg | 150 | mg | 300 | mg | 600 | mg | 900 | mg | 300-600 | mg |
| Slippery elm¹ | 75 | mg | 100 | mg | 200 | mg | 400 | mg | 600 | mg | 200-400 | mg |
| Mucin¹ | 75 | mg | 100 | mg | 200 | mg | 400 | mg | 600 | mg | 200-400 | mg |
| Marshmallow¹ | 25 | mg | 50 | mg | 100 | mg | 200 | mg | 300 | mg | 100-200 | mg |
| Chamomile¹ | 25 | mg | 50 | mg | 100 | mg | 200 | mg | 300 | mg | 100-200 | mg |
| Okra extract¹ | 25 | mg | 50 | mg | 100 | mg | 200 | mg | 300 | mg | 100-200 | mg |
| Cat's claw¹ | 25 | mg | 50 | mg | 100 | mg | 200 | mg | 300 | mg | 100-200 | mg |
| Quercetin¹ | 25 | mg | 50 | mg | 100 | mg | 200 | mg | 300 | mg | 100-200 | mg |
| Sodium butyrate¹ | 25 | mg | 50 | mg | 100 | mg | 200 | mg | 300 | mg | 100-200 | mg |
| Curcumin¹ | 0.25 | g | 0.5 | g | 1 | g | 2 | g | 3 | g | 1-2 | g |
| Mastic gum¹ | 125 | mg | 250 | mg | 400 | mg | 800 | mg | 1.2 | g | 400-800 | mg |
| Saccharomyces boulardii^{1,2} | | | | | | | | | | | | |
| Topical colloidal silver^{3,4} | | | | | | | | | | | | |
| Oral hyaluronic acid³ | 25 | mg | 50 | mg | 100 | mg | 200 | mg | 300 | mg | 100-200 | mg |
| Oral green tea^{5,6} | 1 | cup | 1 | cup | 2 | cups | 2-3 | cups | 3-4 | cups | 2 | cups |
| Colloidal silver nasal spray^{5, 7} | | | | | | | | | | | | |

¹Indicated for GI conditions.
²Indicated for diarrhea. Variable dose starting at 5B CFU, which is titrated up until symptoms abate, then tapered down as symptoms subside.
³Indicated for skin conditions
⁴Provided as a cream, ointment or salve, used according to standard method
⁵Indicated for respiratory conditions.
⁶Provided as a liquid
⁷ Provided as a spray mist, used according to standard method

TABLE 7
Typical daily dosages of ingredients promoting cellular bioenergetics

| | Maintenance | | Stage 1 | | Stage 2 | | Stage 3 | | Stage 4 | | Leaky gut | |
|---|---|---|---|---|---|---|---|---|---|---|---|---|
| Component | Dose | | Dose | | Dose | | Dose | | Dose | | Dose | |
| CoQ10 - ubiquinol | 50 | mg | 100 | mg | 100-200 | mg | 200-400 | mg | 300-600 | mg | 100-200 | mg |
| CoQ10 - ubiquinone¹ | 450 | mg | 900 | mg | 0.9-1.8 | g | 1.8-3.6 | g | 2.7-5.4 | g | 0.9-1.8 | g |
| L-carnitine | 500 | mg | 0.5-1 | g | 1-2 | g | 2-2.5 | g | 2.5-3.5 | g | 1-2.5 | g |
| PQQ | 5 | mg | 10 | mg | 20 | mg | 30 | mg | 40 | mg | 20-30 | mg |
| D-ribose | 5 | g | 5-10 | g | 10-15 | g | 10-15 | g | 15 | g | 10-15 | g |
| Alpha-lipoic acid | 50 | mg | 100 | mg | 200 | mg | 400 | mg | 600 | mg | 200-400 | mg |
| Magnesium² | 400 | mg | 400-800 | mg | 400-800 | mg | 400-800 | mg | 400-800 | mg | 400-800 | mg |

¹Optional, alternative to CoQ10 - ubiquinol.
²Contraindicated for diarrhea.

TABLE 8
Typical daily dosages of nutrients promoting tissue healing

| | Maintenance | | Stage 1 | | Stage 2 | | Stage 3 | | Stage 4 | | Leaky gut | |
|---|---|---|---|---|---|---|---|---|---|---|---|---|
| Component | Dose | | Dose | | Dose | | Dose | | Dose | | Dose | |
| L-arginine¹ | 0.5 | g | 0.75 | g | 15 | g | 3 | g | 4.5 | g | 1.5-3 | g |
| Hawthorne berry¹ | 125 | mg | 250 | mg | 400 | mg | 800 | mg | 1.2 | g | 400-800 | mg |
| Garlic extract^{1,2} | 125 | mg | 250 | mg | 400 | mg | 800 | mg | 1.2 | g | 400-800 | mg |
| Milk thistle seed extract² | 0.25 | g | 0.5 | g | 1 | g | 2 | g | 3 | g | 1-2 | g |
| Artichoke extract² | 125 | mg | 250 | mg | 400 | mg | 800 | mg | 1.2 | g | 400-800 | mg |
| Phospholipids² | 150 | mg | 150-300 | mg | 300-600 | mg | 600-1200 | mg | 1.2-2.4 | g | 300-900 | mg |
| Curcumin² | 0.25 | g | 0.5 | g | 1 | g | 2 | g | 3 | g | 1-2 | g |
| Ginkgo biloba³ | 125 | mg | 250 | mg | 400 | mg | 800 | mg | 1.2 | g | 400-800 | mg |
| Vinpocetine³ | 5 | mg | 10 | mg | 20 | mg | 30 | mg | 40 | mg | 20-30 | mg |
| Botanical adaptogens^{4, 5} | 0.5 | g | 0.75 | g | 1.5 | g | 3 | g | 4.5 | g | 1.5-3 | g |

¹Indicated for cardiovascular conditions.
²Indicated for liver conditions.
³Indicated for brain microcirculation support.
⁴Indicated for adrenal conditions.
⁵ Dose is for each of the botanical adaptogens included.

TABLE 9
Typical daily dosages of enzymes that dissolve or reduce scabs, polyps, callouses and/or scars

| | Maintenance | | Stage 1 | | Stage 2 | | Stage 3 | | Stage 4 | | Leaky gut | |
|---|---|---|---|---|---|---|---|---|---|---|---|---|
| Component | Dose | | Dose | | Dose | | Dose | | Dose | | Dose | |
| Pancreatin | 100 | mg | 150 | mg | 300 | mg | 600 | mg | 1 | g | 300-600 | mg |
| Papain Carica papaya (fruit) | 70 | mg | 100 | mg | 180 | mg | 360 | mg | 600 | mg | 180-360 | mg |
| Bromelain Ananas comosus (stem) | 50 | mg | 80 | mg | 135 | mg | 270 | mg | 450 | mg | 135-270 | mg |
| Trypsin | 20 | mg | 40 | mg | 72 | mg | 144 | mg | 250 | mg | 72-144 | mg |
| Chymotrypsin | 1 | mg | 2 | mg | 3 | mg | 6 | mg | 10 | mg | 3-6 | mg |
| Rutin Sophora japonica | 60 | mg | 90 | mg | 150 | mg | 300 | mg | 450 | mg | 150-300 | mg |

In some embodiments, the colostrum is from cow, sheep, goat or human. In some embodiments, the colostrum is from a different animal as the individual to be administered with the composition. In some embodiments, the colostrum is from the same animal as the individual to be administered with the composition. In some embodiments, the colostrum comprises from about 10% to about 30% (such as about any of 10, 15, 20, 25, or 30%, including any ranges between these values) immunoglobulins. In some embodiments, the colostrum comprises about 30% immunoglobulins. In some embodiments, the colostrum is substantially non-allergenic. In some embodiments, the colostrum is low allergenic. In some embodiments, the colostrum is essentially free of allergens, which include, but are not limited to, lactose and milk protein. In some embodiments, the colostrum is provided bound to phospholipid. In some embodiments, the colostrum is collected less than about 48 (such as less than about any of 48, 24, 12 or 6) hours after birthing. In some embodiments, the composition comprises colostrum at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed in Table 3 for colostrum. In some embodiments, the composition comprises colostrum at about one of the specific amounts listed in Table 3 for colostrum.

In some embodiments, the protein component of the composition comprises whey protein. In some embodiments, the whey protein is from cow, sheep or goat. In some embodiments, the whey protein is from cows. In some embodiments, the whey protein is a concentrate. In some embodiments, the whey protein is an isolate. In some embodiments, the whey protein is hydrolyzed. In some embodiments, the whey protein is non-denatured. In some embodiments, the whey protein is non-homogenized. In some embodiments, the whey protein is from cows that are grass-fed. In some embodiments, the whey protein is from cows that are free of bovine spongiform encephalopathy (BSE). In some embodiments, the whey protein has a high amino acid content. In some embodiments, the whey protein has a high immunoglobulin content, including, for example, high levels of immunoglobulin G (IgG) and immunoglobulin A (IgA). In some embodiments, the whey protein contains at least about 2% (such as at least about any of 2%, 4%, 6%, 8%, 10%, or more) by weight immunoglobulin content. Thus in some embodiments, the protein component comprises non-denatured whey protein concentrate from grass-fed cows free of BSE, and contains high levels of amino acids and immunoglobulins, such as IgG and IgA. In an alternative embodiment, for individuals allergic and/or sensitive to milk and/or casein proteins, the protein is a non-dairy protein, including, but not limited to, beef, pea or soy protein. In some embodiments, the composition comprises protein, such as whey or beef protein, at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed in Table 3 for the corresponding ingredient. In some embodiments, the composition comprises protein, such as whey or beef protein, at about one of the specific amounts listed in Table 3 for the corresponding ingredient.

An exemplary amino acid profile of a whey protein concentrate useful for the compositions described herein is: 0.39 g alanine, 0.17 g arginine, 0.86 g aspartic acid, 0.24 g cysteine, 1.51 g glutamic acid, 0.23 g glycine, 0.15 g histidine, 0.44 g isoleucine, 0.71 g leucine, 0.8 g lysine, 0.16 g methionine, 0.25 g phenylalanine, 0.53 g proline, 0.42 g serine, 0.63 g threonine, 0.17 g tryptophan, 0.27 g tyrosine, 0.37 g valine and >12% IgG, per 10 g serving.

In some embodiments, the detoxification component comprises one of a plasma chelation therapy, modified citrus pectin or an oral chelation and antioxidant detox formula. Plasma chelation therapy is administered by a skilled professional. In some embodiments, the oral chelation and antioxidant detox formula comprises at least one (such as at least any of 2, 5, 10, 15, or 19) of: calcium disodium EDTA, N-acetyl cysteine, chlorella, rosemary leaf extract, turmeric root extract, modified citrus pectin, garlic bulb, broccoli sprouts, holy basil leaf extract, olive leaf extract, silymarin, calcium D-glucarate, green tea leaf extract, cilantro leaf extract, wasabi rhizome, shilajit mineral resin extract, R-lipoic acid, coenzyme Q10, or black pepper fruit extract. In some embodiments, the detoxification component further comprises antioxidants.

In some embodiments, the detoxification component comprises plasma chelation therapy. In some embodiments, the detoxification component comprises modified citrus pectin. In some embodiments, the detoxification component comprises modified citrus pectin at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed in Table 4 for modified citrus pectin. In some embodiments, the detoxification component comprises modified citrus pectin at about one of the specific amounts listed in Table 4 for modified citrus pectin. In some embodiments, the detoxification component comprises at least one (such as at least any of 2, 5, 10, 15, or 19) item listed in Table 4 for oral chelation and antioxidant detox formula. In some embodiments, the detoxification component comprises at least one (such as at least any of 2, 5, 10, 15, or 19) item listed in Table 4 for oral chelation and antioxidant detox formula at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the detoxification component comprises at least one (such as at least any of 2, 5, 10, 15, or 19) item listed in Table 4 for oral chelation and antioxidant detox formula at about one of the specific amounts listed for the corresponding items. In some embodiments, the detoxification component comprises at least two (such as at least any of 3, 5, 10, 15, or 19) items listed in Table 4 for oral chelation and antioxidant detox formula at the same relative weight ratios as the weight ratios calculated based on the specific amounts listed for the corresponding items. In some embodiments, the detoxification component comprises all the items listed in Table 4 for oral chelation and antioxidant detox formula. In some embodiments, the detoxification component comprises all the items listed in Table 4 for oral chelation and antioxidant detox formula at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the detoxification component comprises all the items listed in Table 4 for oral chelation and antioxidant detox formula at about one of the specific amounts listed for the corresponding items. In some embodiments, the detoxification component comprises all the items listed in Table 4 for oral chelation and antioxidant detox formula at the same relative weight ratios as the weight ratios calculated based on the specific amounts listed for the corresponding items. In some embodiments, the detoxification component further comprises antioxidants. In some embodiments, the antioxidants comprise a recycling antioxidant blend such as Rejuvenation Science® Green C™.

Thus, in some embodiments, there is provided a composition (such as an oral composition) comprising a probiotic component according to any of the embodiments described above, colostrum according to any of the embodiments described above, a protein component according to any of the embodiments described above, and a detoxification component according to any of the embodiments described above.

In some embodiments, the prebiotic component comprises a fiber, such as soluble fiber or insoluble fiber. In some embodiments, the prebiotic component comprises an oligosaccharide. In some embodiments, the fiber is not digestable by a human enzyme but digestible by a bacterium. In some embodiments, the prebiotic component comprises one or more ingredients selected from the group consisting of inulin, galactooligosaccharide (GOS), fructooligosaccharide (FOS), xylooligosaccharide (XOS), Mannan-oligosaccharide (MOS) and polydextrose. In some embodiments, the prebiotic component comprises one ingredient selected from the group consisting of inulin, galactooligosaccharide (GOS), fructooligosaccharide (FOS), xylooligosaccharide (XOS), Mannan-oligosaccharide (MOS) and polydextrose. In some embodiments, the prebiotic component comprises GOS and FOS. In some embodiments, the prebiotic component comprises inulin and GOS. In some embodiments, the prebiotic component comprises inulin and FOS. In some embodiments, the prebiotic component comprises inulin, GOS, and FOS. In some embodiments, the prebiotic component comprises inulin, GOS, and FOS at the relative ratio of: about 1:1:1, about 100:1:1, about 1:100:1, about 1:1:100, about 100:100:1, about 100:1:100, or about 1:100:100, or any range in between these values. In some embodiments, the total amount of the ingredients in the prebiotic component is from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed in Table 3. In some embodiments, the total amount of the ingredients in the prebiotic component is one of the specific amounts listed in Table 3.

The compositions described herein may comprise a tissue constituent component. In some embodiments, the tissue constituent component comprises an organic constituent of a tissue which includes, but is not limited to, GI, nasal, lung, liver, mouth, vagina, urinary tract, bladder, ear, eye, and neural tissue. In some embodiments, the organic constituent of a tissue is an organic constituent of the epithelial, fat, or muscle lining. In some embodiments, the organic constituent of a tissue is an organic constituent of the sinus, lungs, liver, ears, vagina, eyes, urethra, kidney, bladder, skin or brain.

In some embodiments, the tissue constituent component comprises at least one (such as at least any of 2, 3, 4 or 5) of: lipid replacement formula, multivitamin (such as Rejuvenation Science Maximum Vitality), methylsulfonylmethane (MSM) and vitamin D3. In some embodiments, the lipid replacement formula comprises omega-3 polyunsaturated fatty acids (PUFAs), phospholipids, amino acids, L-glutamine and N-acetyl glucosamine. In some embodiments, the lipid replacement formula comprises oil which is high in both omega-3 fatty acids and phospholipids, such as oil from crustaceans or mollusks. In some embodiments, the lipid replacement formula comprises krill or green-lipped mussel oil as a source of omega-3 PUFAs and phospholipids. In an alternative embodiment, the lipid replacement formula comprises fish oil as a source of omega-3 PUFAs and a phospholipid complex comprising phosphatidyl serine, phosphatidyl choline, phosphatidyl ethanolamine, phosphatidyl inositol, phosphatidic acid and acetylated phosphatidyl ethanolamine as a source of phospholipids. In some embodiments, where the composition is used in a method of treating a GI condition, the tissue constituent component further comprises at least one (such as at least any of 1, 2 or 3) of: zinc carnosine, mucin and sodium butyrate. In some embodiments, where the composition is used in a method of treating a cardiovascular condition, the tissue constituent component further comprises at least one (such as at least any of 1, 2, or 3) of: magnesium, taurine and L-arginine. In some embodiments, where the composition is used in a method of treating a skin condition, the tissue constituent component further comprises at least one (such as at least 1 or 2) of: MSM and silica. In some embodiments, where the composition is used in a method of treating a nervous system condition, the tissue constituent component further comprises at least one (such as at least any of 1, 2 or 3) of: phosphatidyl serine, dimethylaminoethanol (DMAE) and niacinamide. In some embodiments, where the composition is used in a method of treating a joint condition, the tissue constituent component further comprises at least one (such as at least any of 1, 2, 3, 4 or 5) of: cetyl myristoleate (CMO), hyaluronic acid, glucosamine, chondroitin and MSM.

In some embodiments, the tissue constituent component comprises at least one (such as at least any of 2, 5, 10, 15, 20, or more) item from Table 5. In some embodiments, the organic constituent of a tissue comprises at least one (such as at least any of 2, 5, 10, 15, 20, or more) item from Table 5 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue constituent component comprises at least one (such as at least any of 2, 5, 10, 15, 20, or more) item from Table 5 at about one of the specific amounts listed for the corresponding items. In some embodiments, the tissue constituent component comprises at least two (such as at least any of 3, 5, 10, 15, 20, or more) items from Table 5 at the same relative weight ratios as the weight ratios calculated based on the specific amounts listed for the corresponding items. In some embodiments, the tissue constituent component comprises all of the non-alternative items listed in Table 5. In some embodiments, the tissue constituent component comprises all of the non-alternative items listed in Table 5 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue constituent component comprises all of the non-alternative items listed in Table 5 at about one of the specific amounts listed for the corresponding items. In some embodiments, the tissue constituent component comprises all of the non-alternative items listed in Table 5 at the same relative weight ratios as the weight ratios calculated based on the specific amounts listed for the corresponding items. In some embodiments, where the tissue constituent component comprises vitamin D3, the amount of the vitamin D3 is titrated to achieve a plasma level of 80-100 ng/ml 25-hydroxyvitamin D [25(OH)D] (typically about 5,000 to about 10,000 IU/day) in the individual to which the compositions is administered.

“Non-alternative” used herein when referring to items listed in a table means any item that is not indicated in the table as an alternative to another item listed in the table.

In some embodiments, the tissue constituent component comprises all of the non-indicated and GI-indicated items listed in Table 5. In some embodiments, the tissue constituent component comprises all of the non-indicated and GI-indicated items listed in Table 5 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue constituent component comprises all of the non-indicated and GI-indicated items listed in Table 5 at about one of the specific amounts listed for the corresponding items. In some embodiments, the tissue constituent component comprises all of the non-indicated and GI-indicated items listed in Table 5 at the same relative weight ratios as the weight ratios calculated based on the specific amounts listed for the corresponding items. In some embodiments, the amount of the vitamin D3 is titrated to achieve a plasma level of 80-100 ng/ml 25-hydroxyvitamin D [25(OH)D] (typically about 5,000 to about 10,000 IU/day) in the individual to which the compositions is administered.

“Non-indicated” used herein when referring to items listed in a table means any item that is not indicated for a particular condition or tissue in the table and is not an alternative item.

In some embodiments, the tissue constituent component comprises all of the non-indicated and cardiovascular-indicated items listed in Table 5. In some embodiments, the tissue constituent component comprises all of the non-indicated and cardiovascular-indicated items listed in Table 5 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue constituent component comprises all of the non-indicated and cardiovascular-indicated items listed in Table 5 at about one of the specific amounts listed for the corresponding items. In some embodiments, the tissue constituent component comprises all of the non-indicated and cardiovascular-indicated items listed in Table 5 at the same relative weight ratios as the weight ratios calculated based on the specific amounts listed for the corresponding items. In some embodiments, the amount of the vitamin D3 is titrated to achieve a plasma level of 80-100 ng/ml 25-hydroxyvitamin D [25(OH)D] (typically about 5,000 to about 10,000 IU/day) in the individual to which the compositions is administered.

In some embodiments, the tissue constituent component comprises all of the non-indicated and skin-indicated items listed in Table 5. In some embodiments, the tissue constituent component comprises all of the non-indicated and skin-indicated items listed in Table 5 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue constituent component comprises all of the non-indicated and skin-indicated items listed in Table 5 at about one of the specific amounts listed for the corresponding items. In some embodiments, the tissue constituent component comprises all of the non-indicated and skin-indicated items listed in Table 5 at the same relative weight ratios as the weight ratios calculated based on the specific amounts listed for the corresponding items. In some embodiments, the amount of the vitamin D3 is titrated to achieve a plasma level of 80-100 ng/ml 25-hydroxyvitamin D [25(OH)D] (typically about 5,000 to about 10,000 IU/day) in the individual to which the compositions is administered.

In some embodiments, the tissue constituent component comprises all of the non-indicated and brain-indicated items listed in Table 5. In some embodiments, the tissue constituent component comprises all of the non-indicated and brain-indicated items listed in Table 5 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue constituent component comprises all of the non-indicated and brain-indicated items listed in Table 5 at about one of the specific amounts listed for the corresponding items. In some embodiments, the tissue constituent component comprises all of the non-indicated and brain-indicated items listed in Table 5 at the same relative weight ratios as the weight ratios calculated based on the specific amounts listed for the corresponding items. In some embodiments, the amount of the vitamin D3 is titrated to achieve a plasma level of 80-100 ng/ml 25-hydroxyvitamin D [25(OH)D] (typically about 5,000 to about 10,000 IU/day) in the individual to which the compositions is administered.

In some embodiments, the tissue constituent component comprises all of the non-indicated and joint-indicated items listed in Table 5. In some embodiments, the tissue constituent component comprises all of the non-indicated and joint-indicated items listed in Table 5 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue constituent component comprises all of the non-indicated and joint-indicated items listed in Table 5 at about one of the specific amounts listed for the corresponding items. In some embodiments, the tissue constituent component comprises all of the non-indicated and joint-indicated items listed in Table 5 at the same relative weight ratios as the weight ratios calculated based on the specific amounts listed for the corresponding items. In some embodiments, the amount of the vitamin D3 is titrated to achieve a plasma level of 80-100 ng/ml 25-hydroxyvitamin D [25(OH)D] (typically about 5,000 to about 10,000 IU/day) in the individual to which the compositions is administered.

In some embodiments, according to any of the components comprising an organic constituent of a tissue described above comprising oil from a crustacean or mollusk, such as krill or green-lipped mussel oil, the oil is substituted with fish oil and phospholipids.

The compositions described herein may comprise a symptomatic relief component. In some embodiments, the symptomatic relief component comprises at least one (such as at least any of 2, 3, 4, 5, 10, 15 or 17) of: Citrus Pectin, Deglycyrrhizinated Licorice (DGL), Aloe Vera Extract (Aloe barbadensis) (leaf), Slippery Elm (Ulmus fulva) (bark), Mucin, Marshmallow (Althaea officinalis) (root), Chamomile (Matricaria chamomilla) (flower), Okra Extract (Abelmoschus esculentus) (fruit), Cat's Claw (Uncaria tomentosa) (bark), Quercetin, Sodium Butyrate, Curcumin, mastic gum, colloidal silver, hyaluronic acid, green tea and Saccharomyces boulardii. In some embodiments, where the composition is used in a method of treating a condition of the GI tract, the symptomatic relief component comprises at least one (such as at least any of 2, 3, 4, 5, 6, 7, 8 or 9) of: Deglycyrrhizinated Licorice (DGL), Aloe Vera Extract (Aloe barbadensis) (leaf), Slippery Elm (Ulmus fulva) (bark), Marshmallow (Althaea officinalis) (root), Chamomile (Matricaria chamomilla) (flower), Okra Extract (Abelmoschus esculentus) (fruit), Cat's Claw (Uncaria tomentosa) (bark), Quercetin, Curcumin and Mastic Gum. In some embodiments, where the composition is used in a method of treating a condition associated with diarrhea, the symptomatic relief component further comprises Saccharomyces boulardii. In some embodiments, where the composition is used in a method of treating a condition of the skin, the symptomatic relief component comprises at least one (such as at least 1 or 2) of: topical colloidal silver and oral hyaluronic acid. In some embodiments, where the composition is used in a method of treating a respiratory condition, the symptomatic relief component comprises at least one (such as at least 1 or 2) of: oral green tea and colloidal silver nasal spray. In some embodiments, where the composition is used in a method of treating a condition of the skin, the method does not comprise administration of antibiotic or antifungal ingredients. In some embodiments, where the composition is used in a method of treating a respiratory condition, the method does not comprise administration of antibiotic or antifungal ingredients.

In some embodiments, the symptomatic relief component comprises at least one (such as at least any of 2, 5, 10, 15, or 18) item from Table 6. In some embodiments, the symptomatic relief component comprises at least one (such as at least any of 2, 5, 10, 15, or 18) item from Table 6 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the symptomatic relief component comprises at least one (such as at least any of 2, 5, 10, 15, or 18) item from Table 6 at about one of the specific amounts listed for the corresponding items. In some embodiments, the symptomatic relief component comprises at least two (such as at least any of 3, 5, 10, 15, or 18) items from Table 6 at the same relative weight ratios as the weight ratios calculated based on the specific amounts listed for the corresponding items. In some embodiments, the symptomatic relief component comprises all the non-dosage form-indicated items listed in Table 6. In some embodiments, the symptomatic relief component comprises all the non-dosage form-indicated items listed in Table 6 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the symptomatic relief component comprises all the non-dosage form-indicated items listed in Table 6 at about one of the specific amounts listed for the corresponding items. In some embodiments, the symptomatic relief component comprises all the non-dosage form-indicated items listed in Table 6 at the same relative weight ratios as the weight ratios calculated based on the specific amounts listed for the corresponding items. In some embodiments, where the symptomatic relief component comprises Saccharomyces boulardii, the symptomatic relief component comprises Saccharomyces boulardii at a starting dose of 5B CFU, which is titrated up until symptoms abate, and then tapered down as symptoms subside.

“Non-dosage form-indicated” as used herein in reference to an item listed in a table means the item is not indicated as being provided in a particular dosage form (e.g., provided as a cream, liquid, or spray) in the table.

In some embodiments, the symptomatic relief component comprises all the GI-indicated items listed in Table 6. In some embodiments, the symptomatic relief component comprises all the GI-indicated items listed in Table 6 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the symptomatic relief component comprises all the GI-indicated items listed in Table 6 at about one of the specific amounts listed for the corresponding items. In some embodiments, the symptomatic relief component comprises all the GI-indicated items listed in Table 6 at the same relative weight ratios as the weight ratios calculated based on the specific amounts listed for the corresponding items.

In some embodiments, the symptomatic relief component comprises all the GI- and diarrhea-indicated items listed in Table 6. In some embodiments, the symptomatic relief component comprises all the GI- and diarrhea-indicated items listed in Table 6 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the symptomatic relief component comprises all the GI- and diarrhea-indicated items listed in Table 6 at about one of the specific amounts listed for the corresponding items. In some embodiments, the symptomatic relief component comprises all the GI- and diarrhea-indicated items listed in Table 6 at the same relative weight ratios as the weight ratios calculated based on the specific amounts listed for the corresponding items. In some embodiments, the Saccharomyces boulardii in the symptomatic relief component is at a starting dose of 5B CFU, which is titrated up until symptoms abate, and then tapered down as symptoms subside.

The compositions described herein may comprise a cellular bioenergetics component. In some embodiments, the cellular bioenergetics component comprises at least one (such as at least any of 2, 3, 4, 5 or 6) of: Coenzyme Q10 (CoQ10), L-Carnitine, Pyrroloquinoline quinone (PQQ), D-Ribose, alpha-Lipoic Acid and Magnesium. In a preferred embodiment, the CoQ10 is reduced CoQ10, ubiquinol. In an alternative embodiment, the CoQ10 is oxidized CoQ10, ubiquinone. In some embodiments, the CoQ10 is a mixture of ubiquinol and ubiquinone having a ratio of ubiquinol to ubiquinone from about 10:1 to about 1:10 (such as about any of 8:1, 6:1, 4:1, 2:1, 1:1, 1:2, 1:4, 1:6 or 1:8). In a preferred embodiment, the alpha-lipoic acid is R-lipoic acid. In an alternative embodiment, the alpha-lipoic acid is S-lipoic acid. In some embodiments, the alpha-lipoic acid is a mixture of R-lipoic acid and S-lipoic acid having a ratio of R-lipoic acid to S-lipoic acid from about 10:1 to about 1:10 (such as about any of 8:1, 6:1, 4:1, 2:1, 1:1, 1:2, 1:4, 1:6 or 1:8). In some embodiments, where the composition is used in a method of treating a condition of the nervous system, the L-carnitine is acetyl L-carnitine.

In some embodiments, the cellular bioenergetics component comprises at least one (such as at least any of 2, 3, 4, 5, or 6) item listed in Table 7. In some embodiments, the cellular bioenergetics component comprises at least one (such as at least any of 2, 3, 4, 5, or 6) item from Table 7 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the cellular bioenergetics component comprises at least one (such as at least any of 2, 3, 4, 5, or 6) item from Table 7 at about one of the specific amounts listed for the corresponding items. In some embodiments, the cellular bioenergetics component comprises at least two (such as at least any of 3, 4, 5, or 6) items from Table 7 at the same relative weight ratios as the weight ratios calculated based on the specific amounts listed for the corresponding items. In some embodiments, the cellular bioenergetics component comprises all the non-alternative items listed in Table 7. In some embodiments, the cellular bioenergetics component comprises all the non-alternative items listed in Table 7 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the cellular bioenergetics component comprises all the non-alternative items listed in Table 7 at about one of the specific amounts listed for the corresponding items. In some embodiments, the cellular bioenergetics component comprises all the non-alternative items listed in Table 7 at the same relative weight ratios as the weight ratios calculated based on the specific amounts listed for the corresponding items.

In some embodiments, the cellular bioenergetics component comprises all the non-contraindicated items listed in Table 7. In some embodiments, the cellular bioenergetics component comprises all the non-contraindicated items listed in Table 7 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the cellular bioenergetics component comprises all the non-contraindicated items listed in Table 7 at about one of the specific amounts listed for the corresponding items. In some embodiments, the cellular bioenergetics component comprises all the non-contraindicated items listed in Table 7 at the same relative weight ratios as the weight ratios calculated based on the specific amounts listed for the corresponding items.

“Non-contraindicated” used herein when referring to items listed in a table means any item that is not contraindicated for a particular condition or tissue in the table and is not an alternative item.

In some embodiments, according to any of the components comprising a nutrient that promotes cellular bioenergetics described above comprising CoQ10-ubiquinol, the CoQ10-ubiquinol is substituted with CoQ10-ubiquinone.

The compositions described herein may comprise a tissue healing component. In some embodiments, the tissue healing component comprises at least one (such as at least any of 2, 3, 4, 5, 10, 15, 20 or 25) of: L-Arginine, hawthorne berry, garlic, milk thistle seed extract, artichoke extract, phospholipids, curcumin, garlic extract, Ginkgo biloba, vinpocetine, and botanical adaptogens. In some embodiments, where the composition is used in a method of treating a cardiovascular condition, the tissue healing component comprises at least one (such as at least 1, 2 or 3) of: L-Arginine, hawthorne berry and garlic. In some embodiments, where the composition is used in a method of treating a condition of the liver, the tissue healing component comprises at least one (such as at least 1, 2, 3, 4 or 5) of: milk thistle seed extract, artichoke extract, phospholipids, curcumin and garlic extract. In some embodiments, where the composition is used in a method comprising providing brain microcirculation support, the tissue healing component comprises at least one (such as at least 1 or 2) of: gingko biloba and vinpocetine. In some embodiments, where the composition is used in a method of treating an adrenal condition, the tissue healing component comprises at least one (such as at least 2, 3, 4, 5, 6, 7, 8, 9 or 10) botanical adaptogen. In some embodiments, the botanical adaptogen is selected from the group consisting of schisandra fruit, Chinese yam rhizome, poria sclerotium, Asian water plantain rhizome, rehmannia root, Chinese dodder seed, Asian plantain seed, palm-leaf raspberry fruit, lycium fruit extract and Asiatic dogwood berry extract. In some embodiments, the lycium fruit extract is lycium fruit extract (4:1). In some embodiments, the Asiatic dogwood berry extract is Asiatic dogwood berry extract (5:1).

In some embodiments, the tissue healing component comprises at least one (such as at least any of 2, 5, 10, 15, 20, or more) item listed in Table 8. In some embodiments, the tissue healing component comprises at least one (such as at least any of 2, 5, 10, 15, 20, or more) item listed in Table 8 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue healing component comprises at least one (such as at least any of 2, 5, 10, 15, 20, or more) item listed in Table 8 at about one of the specific amounts listed for the corresponding items. In some embodiments, the tissue healing component comprises at least two (such as at least any of 3, 5, 10, 15, 20, or more) items listed in Table 8 at the same relative weight ratios as the weight ratios calculated based on the specific amounts listed for the corresponding items. In some embodiments, the tissue healing component comprises all the items listed in Table 8. In some embodiments, the tissue healing component comprises all the items listed in Table 8 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue healing component comprises all the items listed in Table 8 at about one of the specific amounts listed for the corresponding items. In some embodiments, the tissue healing component comprises all the items listed in Table 8 at the same relative weight ratios as the weight ratios calculated based on the specific amounts listed for the corresponding items.

In some embodiments, the tissue healing component comprises all the cardiovascular-indicated items listed in Table 8. In some embodiments, the tissue healing component comprises all the cardiovascular-indicated items listed in Table 8 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue healing component comprises all the cardiovascular-indicated items listed in Table 8 at about one of the specific amounts listed for the corresponding items. In some embodiments, the tissue healing component comprises all the cardiovascular-indicated items listed in Table 8 at the same relative weight ratios as the weight ratios calculated based on the specific amounts listed for the corresponding items.

In some embodiments, the tissue healing component comprises all the liver-indication items listed in Table 8. In some embodiments, the tissue healing component comprises all the liver-indication items listed in Table 8 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue healing component comprises all the liver-indication items listed in Table 8 at about one of the specific amounts listed for the corresponding items. In some embodiments, the tissue healing component comprises all the liver-indication items listed in Table 8 at the same relative weight ratios as the weight ratios calculated based on the specific amounts listed for the corresponding items.

In some embodiments, the tissue healing component comprises all the brain microcirculation-indicated items listed in Table 8. In some embodiments, the tissue healing component comprises all the brain microcirculation-indicated items listed in Table 8 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue healing component comprises all the brain microcirculation-indicated items listed in Table 8 at about one of the specific amounts listed for the corresponding items. In some embodiments, the tissue healing component comprises all the brain microcirculation-indicated items listed in Table 8 at the same relative weight ratios as the weight ratios calculated based on the specific amounts listed for the corresponding items.

In some embodiments, the tissue healing component comprises all the adrenal-indicated items listed in Table 8. In some embodiments, the tissue healing component comprises all the adrenal-indicated items listed in Table 8 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the tissue healing component comprises all the adrenal-indicated items listed in Table 8 at about one of the specific amounts listed for the corresponding items. In some embodiments, the tissue healing component comprises all the adrenal-indicated items listed in Table 8 at the same relative weight ratios as the weight ratios calculated based on the specific amounts listed for the corresponding items.

The compositions described herein may comprise an enzyme component. In some embodiments, the enzyme component comprises at least one (such as at least any of 1, 2, 3, 4, 5, or 6) of: Pancreatin. Papain Carica papaya (fruit), Bromelain Ananas comosus (stem), Trypsin, Chymotrypsin, and Rutin Sophora japonica.

In some embodiments, the enzyme component comprises at least one (such as at least any of 2, 3, 4, 5, or 6) item listed in Table 9. In some embodiments, the enzyme component comprises at least one (such as at least any of 2, 3, 4, 5, or 6) item listed in Table 9 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the enzyme component comprises at least one (such as at least any of 2, 3, 4, 5, or 6) item listed in Table 9 at about one of the specific amounts listed for the corresponding items. In some embodiments, the enzyme component comprises at least two (such as at least any of 3, 4, 5, or 6) items listed in Table 9 at the same relative weight ratios as the weight ratios calculated based on the specific amounts listed for the corresponding items. In some embodiments, the enzyme component comprises all the items listed in Table 9. In some embodiments, the enzyme component comprises all the items listed in Table 9 at from about 0.1 to about 100 times (including for example about 0.25 to about 20 times, about 0.5 to about 10 times, or about any of 0.25×, 0.5×. 1×, 5×, 10×, 15×, or 20×) one of the specific amounts listed for the corresponding items. In some embodiments, the enzyme component comprises all the items listed in Table 9 at about one of the specific amounts listed for the corresponding items. In some embodiments, the enzyme component comprises all the items listed in Table 9 at the same relative weight ratios as the weight ratios calculated based on the specific amounts listed for the corresponding items.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and at least one (such as at least 1, 2, 3, 4, 5, or 6) of a prebiotic component according to any of the embodiments described above, a tissue constituent component according to any of the embodiments described above, a symptomatic relief component according to any of the embodiments described above, a cellular bioenergetics component according to any of the embodiments described above, a tissue healing component according to any of the embodiments described above, and an enzyme component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and a prebiotic component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and a tissue constituent component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and a symptomatic relief component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and a cellular bioenergetics component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and a tissue healing component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and an enzyme component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and two of a prebiotic component according to any of the embodiments described above, a tissue constituent component according to any of the embodiments described above, a symptomatic relief component according to any of the embodiments described above, a cellular bioenergetics component according to any of the embodiments described above, a tissue healing component according to any of the embodiments described above, and an enzyme component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and three of a prebiotic component according to any of the embodiments described above, a tissue constituent component according to any of the embodiments described above, a symptomatic relief component according to any of the embodiments described above, a cellular bioenergetics component according to any of the embodiments described above, a tissue healing component according to any of the embodiments described above, and an enzyme component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and four of a prebiotic component according to any of the embodiments described above, a tissue constituent component according to any of the embodiments described above, a symptomatic relief component according to any of the embodiments described above, a cellular bioenergetics component according to any of the embodiments described above, a tissue healing component according to any of the embodiments described above, and an enzyme component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, and five of a prebiotic component according to any of the embodiments described above, a tissue constituent component according to any of the embodiments described above, a symptomatic relief component according to any of the embodiments described above, a cellular bioenergetics component according to any of the embodiments described above, a tissue healing component according to any of the embodiments described above, and an enzyme component according to any of the embodiments described above.

In some embodiments, there is provided a composition (such as an oral composition) comprising a probiotic component according to any of the embodiments described above, colostrum according to any of the embodiments described above, a protein component according to any of the embodiments described above, a detoxification component according to any of the embodiments described above, a prebiotic component according to any of the embodiments described above, a tissue constituent component according to any of the embodiments described above, a symptomatic relief component according to any of the embodiments described above, a cellular bioenergetics component according to any of the embodiments described above, a tissue healing component according to any of the embodiments described above, and an enzyme component according to any of the embodiments described above.

The compositions described herein can be provided in various dosage forms, which include, but are not limited to, capsules, tablets, soft gels, powder, mist, drops, topical cream, ointment, salve, enema, vaginal or anal suppository, liquid solution or a combination thereof. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to using in a method as described below in order to minimize the decreased viability of the microorganisms after exposure to moisture.

The compositions described herein can be formulated for various delivery routes, which include, but are not limited to, oral, topical, nasal, rectal, vaginal, parenteral (such as intravenous), or a combination thereof. In some embodiments, compositions can be provided in capsule, tablet or soft gel form for oral delivery, in topical cream, ointment or salve form for topical delivery, in mist form for nasal delivery, in suppository form for vaginal and/or rectal delivery or in liquid solution form for parenteral (such as intravenous) delivery.

In some embodiments, the composition comprises about 1% to 90%, including for example about 1% to about 10%, about 10% to about 20%, about 20% to about 30%, about 30% to about 40%, about 40% to about 50%, about 50% to about 60%, about 60% to about 70%, about 70% to about 80% or about 80% to about 90% of the core components (probiotic component, colostrum, protein component and detoxification component) by weight of the composition. In some embodiments, the composition comprises at least about 5%, including for example at least about any of 10%, 15%, 20%, 30%, 40%, 50%, 60%, 70%, 80% or 90% of the core components by weight of the composition. In some embodiments, the composition comprises 100% of the core components by weight of the composition.

In some embodiments, the combination of the probiotic component and the colostrum represent at least about 90%, including for example at least about 95%, 98%, or 99% of the core components by weight. In some embodiments, the weight ratio of the probiotic component and the colostrum in the composition is about 10:1 to about 1:10, including for example about 5:1 to about 1:5, about 3:1 to about 1:3, about 2:1 to about 1:2, or about 1:1.

In some embodiments, the composition comprises about 1% to 20%, including for example about 1% to about 5%, about 5% to about 10%, about 10% to about 15%, or about 15% to about 20% of the tissue constituent component by weight of the composition. In some embodiments, the composition comprises at least about 5%, including for example at least about any of 10%, 15%, 20%, 30%, 40%, 50%, 60%, 70%, 80% or 90% of the tissue constituent component by weight of the composition.

In some embodiments, the composition comprises about 1% to 20%, including for example about 1% to about 5%, about 5% to about 10%, about 10% to about 15%, or about 15% to about 20% of the symptomatic relief component by weight of the composition. In some embodiments, the composition comprises at least about 5%, including for example at least about any of 10%, 15%, 20%, 30%, 40%, 50%, 60%, 70%, 80% or 90% of the symptomatic relief component by weight of the composition.

In some embodiments, the composition comprises about 1% to 20%, including for example about 1% to about 5%, about 5% to about 10%, about 10% to about 15%, or about 15% to about 20% of the cellular bioenergetics component by weight of the composition. In some embodiments, the composition comprises at least about 5%, including for example at least about any of 10%, 15%, 20%, 30%, 40%, 50%, 60%, 70%, 80% or 90% of the cellular bioenergetics component by weight of the composition.

In some embodiments, the composition comprises about 1% to 20%, including for example about 1% to about 5%, about 5% to about 10%, about 10% to about 15%, or about 15% to about 20% of the tissue healing component by weight of the composition. In some embodiments, the composition comprises at least about 5%, including for example at least about any of 10%, 15%, 20%, 30%, 40%, 50%, 60%, 70%, 80% or 90% of the tissue healing component by weight of the composition.

In some embodiments, the composition comprises about 1% to 20%, including for example about 1% to about 5%, about 5% to about 10%, about 10% to about 15%, or about 15% to about 20% of the enzyme component by weight of the composition. In some embodiments, the composition comprises at least about 5%, including for example at least about any of 10%, 15%, 20%, 30%, 40%, 50%, 60%, 70%, 80% or 90% of the enzyme component by weight of the composition.

### Methods of the Present Invention

The present application in some aspects provides methods comprising administering to an individual a composition or plurality of compositions as described herein. In some embodiments, there is provided a method of enhancing, protecting or repairing the integrity of a tissue barrier adjacent to or on a tissue of an individual, comprising administering (such as orally administering) to the individual an effective amount of a composition or plurality of compositions comprising a probiotic component, colostrum, a protein component and a detoxification component. In some embodiments of the method, the composition or plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the composition or plurality of compositions are provided according to any of the embodiments described above for compositions of the present invention.

“Integrity” used herein when referring to a tissue barrier includes the structural integrity of the tissue barrier, the balance of the microorganisms in the tissue barrier, their reproduction and turnover capabilities, their environmental adaptability, their adhesion capabilities within the host, and their intra- and extra-species signaling capabilities.

In some embodiments, there is provided a method of preventing or treating inflammation of a tissue of an individual, comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of preventing or repairing damage (such as tissue damage) in an individual, comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the damage (such as tissue damage) is associated with a disturbance in the integrity of one or more tissue barriers in the individual. In some embodiments, the damage (such as tissue damage) is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the damage (such as tissue damage) is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the damage (such as tissue damage) is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of mitigating leakage of a tissue barrier and/or a mucosal membrane adjacent to or on a tissue of an individual, comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of reducing the risk of a disease or condition in an individual, comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the disease or condition is associated with a disturbance in the integrity of one or more tissue barriers and/or mucosal membranes adjacent to or on one or more tissues of the individual. In some embodiments, the disease or condition is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the disease or condition is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the disease or condition is associated with a disturbance in the integrity of one or more tissue barriers and/or mucosal membranes adjacent to or on one or more tissues of the individual that provide protection from the external environment and disturbance in the integrity of one or more tissue barriers and/or mucosal membranes adjacent to or on one or more tissues of the individual that provide protection from the internal environment. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual has one or more risk factors associated with the disease or condition. In some embodiments, the individual has had one or more previous occurrences of the disease or condition. In some embodiments, the individual is human.

In some embodiments, there is provided a method of reducing a side effect associated with the administration of an antibiotic in an individual, comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the side effect associated with the administration of an antibiotic is associated with a disturbance in the integrity of one or more tissue barriers and/or mucosal membranes adjacent to or on one or more tissues of the individual. In some embodiments, the side effect associated with the administration of an antibiotic is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the side effect associated with the administration of an antibiotic is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the side effect associated with the administration of an antibiotic is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of reducing an adverse effect associated with the exposure of an individual to an environmental contaminant or non-natural substance, comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of reducing an adverse effect associated with abnormal levels of a naturally occurring substance in an individual, comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the adverse effect associated with abnormal levels of a naturally occurring substance is associated with a disturbance in the integrity of one or more tissue barriers and/or mucosal membranes adjacent to or on one or more tissues of the individual. In some embodiments, the adverse effect associated with abnormal levels of a naturally occurring substance is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the adverse effect associated with abnormal levels of a naturally occurring substance is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the adverse effect associated with abnormal levels of a naturally occurring substance is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the naturally occurring substance is a substance produced by the individual. In some embodiments, the naturally occurring substance is present in the individual in abnormal levels systemically and/or locally. For example, in some embodiments, the naturally occurring substance is present in the individual's blood at a level higher than the normal level found in the blood of a healthy individual, or the naturally occurring substance is present in a particular tissue or organ of the individual at a level higher than the normal level found in the tissue or organ, respectively, of a healthy individual. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of simultaneously treating comorbid conditions in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the comorbid conditions are associated with a disturbance in the integrity of one or more tissue barriers and/or mucosal membranes adjacent to or on one or more tissues of the individual. In some embodiments, the comorbid conditions are associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the comorbid conditions are associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the comorbid conditions are associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of simultaneously enhancing, protecting or repairing the integrity of multiple tissue barriers located in different regions in the body of an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of enhancing, protecting or repairing the integrity of the blood-brain barrier in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of preventing or treating a disease associated with brain or neurological dysfunction in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the disease associated with brain or neurological dysfunction is associated with a disturbance in the integrity of one or more tissue barriers and/or mucosal membranes adjacent to or on one or more tissues of the individual. In some embodiments, the disease associated with brain or neurological dysfunction is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the disease associated with brain or neurological dysfunction is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the disease associated with brain or neurological dysfunction is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual has one or more risk factors associated with the disease. In some embodiments, the individual has had one or more previous occurrences of the disease. In some embodiments, the individual is human.

In some embodiments, there is provided a method of reducing the risk of one or more diseases associated with inflammation in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the diseases associated with inflammation are associated with a disturbance in the integrity of one or more tissue barriers and/or mucosal membranes adjacent to or on one or more tissues of the individual. In some embodiments, the diseases associated with inflammation are associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the diseases associated with inflammation are associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the diseases associated with inflammation are associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the diseases associated with inflammation are selected from the group consisting of cardiovascular disease, food allergies, and sinus allergies. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual has one or more risk factors associated with the disease. In some embodiments, the individual has had one or more previous occurrences of the disease. In some embodiments, the individual is human.

In some embodiments, there is provided a method of preventing or treating diseases associated with exposure to one or more contaminants in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the diseases associated with exposure to one or more contaminants are associated with a disturbance in the integrity of one or more tissue barriers and/or mucosal membranes adjacent to or on one or more tissues of the individual. In some embodiments, the diseases associated with exposure to one or more contaminants are associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the diseases associated with exposure to one or more contaminants are associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the diseases associated with exposure to one or more contaminants are associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the contaminants are selected from the group consisting of chlorine, herbicides, pesticides, antibiotics in foods and drinking or bathing water, hydrocarbons, tar, nicotine, smoke, and heavy metals. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual has one or more risk factors associated with the disease. In some embodiments, the individual has had one or more previous occurrences of the disease. In some embodiments, the individual is human.

In some embodiments, there is provided a method of protecting a tissue after surgery and promoting post-surgical healing of the tissue in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the tissue is GI tissue. In some embodiments, the surgery is GI surgery. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of preventing or treating a disease or condition in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the disease or condition is associated with a disturbance in the integrity of a tissue barrier and/or mucosal membrane adjacent to or on a tissue of the individual. In some embodiments, the disease or condition is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the disease or condition is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the disease or condition is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual has one or more risk factors associated with the disease or condition. In some embodiments, the individual has had one or more previous occurrences of the disease or condition. In some embodiments, the individual is human.

In some embodiments, there is provided a method of preventing or treating a GI condition in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for GI conditions. In some embodiments, for each component in the composition, the component comprises all of the ingredients listed for the component in Tables 3-9 indicated for GI conditions. In some embodiments, for each component in the composition, the component comprises at least one ingredient listed for the component in Tables 3-9 indicated for diarrhea. In some embodiments, for each component in the composition, the component comprises all of the ingredients listed for the component in Tables 3-9 indicated for diarrhea. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the GI condition is selected from the group consisting of irritable bowel syndrome (IBS), inflammatory bowel disease (IBD), colitis, ulcerative colitis, Crohn's disease, diverticulitis, acid reflux, heartburn, gastroesophageal reflux disease (GERD), pouchitis and post-surgical healing. In some embodiments, the individual has one or more risk factors associated with the condition. In some embodiments, the individual has had one or more previous occurrences of the condition. In some embodiments, the individual is human.

In some embodiments, there is provided a method of preventing or treating an autoimmune disease in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the autoimmune disease is selected from the group consisting of acute disseminated encephalomyelitis (ADEM), Addison's disease, allergic granulomatosis and angiitis, alopecia or alopecia areata (AA), ankylosing spondylitis, autoimmune chronic active hepatitis (CAH), autoimmune hemolytic anemia, autoimmune pancreatitis (AIP), autoimmune retinopathy (AR), autoimmune thrombocytopenic purpura, autoimmune neutropenia, autoimmune inner ear disease (AIED), antiphospholipid syndrome (APS), autoimmune lymphoproliferative syndrome (ALPS), Behcet's syndrome, bullus pemphigoid, celiac disease, Cogan's syndrome, Churg-Strauss syndrome (CSS), chronic bullous disease of childhood, chronic inflammatory demyelinating polyradiculoneuropathy (CIDP), cictricial pemphigoid (CP), central nervous system vasculitis, Crohn's disease, cryoglobulinemia, dermatitis herpetiformis (DH), dermatomyositis, discoid lupus erythematosus, encephalomyelitis, epidermolysis bullosa acquisita (EBA), erythema nodosum, Evans syndrome, fibromyalgia, giant cell arteritis, graft-versus-host disease, Graves' disease, Gullain-Barre syndrome, Hanot syndrome, Hashimoto's thyroiditis, hypersensitivity vasculitis (HV), inflammatory bowel disease, insulin-dependent diabetes mellitus (type I diabetes), isolated vasculitis of the central nervous system, Isaacs' syndrome, Kawasaki disease (KD), Lambert-Eaton myasthenic syndrome (LEMS), linear IgA disease, lupus, Meniere's disease, microscopic polyangiitis, mixed connective tissue disease or MCTD, monoclonal gammopathy, myasthenia gravis, multiple sclerosis, multifocal motor neuropathy, neuromyotonia, neutropenia, oophoritis, opsoclonus-myoclonus syndrome, orchitis, paraneoplastic neurologic disorders, pemphigus vulgaris, pemphigus follaceus (PF), pemphigoid gestationis (PG), pernicious anemia, paraneoplastic pemphigus (PNP), polyangiitis, polyarteritis nodosa (PAN), polymyositis, polymyalgia rheumatica, primary biliary cirrhosis (PBC), Primary sclerosing cholangitis (PSC), Psoriasis, Raynaud's phenomenon, recoverin-associated retinopathy (RAR), reactive arthritis, retinopathy, rheumatoid arthritis (RA), sarcoidosis, sclerosing cholangitis see primary sclerosing cholangitis, Sjogren's syndrome, systemic necrotizing vascolitides, stiff man syndrome, systemic lupus erythematosus, systemic sclerosis (scleroderma), temporal arteritis, Takayasu's arteritis, thromboangiitis obliterans, thyroiditis with hypothyroidism, thyroiditis with hyperthyroidism, type I autoimmune polyglandular syndrome (PAS), type II autoimmune polyglandular syndrome, ulcerative colitis, vasculitis and Wegener's granulomatosis. In some embodiments, the individual has one or more risk factors associated with the disease. In some embodiments, the individual has had one or more previous occurrences of the disease. In some embodiments, the individual is human.

In some embodiments, there is provided a method of preventing or treating a cardiovascular disorder in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for cardiovascular conditions. In some embodiments, for each component in the composition, the component comprises all of the ingredients listed for the component in Tables 3-9 indicated for cardiovascular conditions. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the cardiovascular disorder is high blood pressure. In some embodiments, the individual has one or more risk factors associated with the cardiovascular disorder. In some embodiments, the individual has had one or more previous occurrences of the cardiovascular disorder. In some embodiments, the individual is human.

In some embodiments, there is provided a method of preventing or treating a liver disorder in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for liver conditions. In some embodiments, for each component in the composition, the component comprises all of the ingredients listed for the component in Tables 3-9 indicated for liver conditions. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the liver disorder is selected from the group consisting of hepatitis, fibrosis, non-alcoholic fatty liver disease (NAFLD) and cirrhosis. In some embodiments, the individual has one or more risk factors associated with the liver disorder. In some embodiments, the individual has had one or more previous occurrences of the liver disorder. In some embodiments, the individual is human.

In some embodiments, there is provided a method of preventing or treating a respiratory disorder in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for respiratory conditions. In some embodiments, for each component in the composition, the component comprises all of the ingredients listed for the component in Tables 3-9 indicated for respiratory conditions. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the respiratory disorder is selected from the group consisting of asthma, allergies, hay fever, sinusitis, chronic obstructive pulmonary disease (COPD) and emphysema. In some embodiments, the individual has one or more risk factors associated with the respiratory disorder. In some embodiments, the individual has had one or more previous occurrences of the respiratory disorder. In some embodiments, the individual is human.

In some embodiments, there is provided a method of preventing or treating a skin disease in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for skin conditions. In some embodiments, for each component in the composition, the component comprises all of the ingredients listed for the component in Tables 3-9 indicated for skin conditions. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the skin disease is selected from the group consisting of acne, allergies, atopic dermatitis, alopecia, rosacea, eczema and psoriasis. In some embodiments, the individual has one or more risk factors associated with the skin disease. In some embodiments, the individual has had one or more previous occurrences of the skin disease. In some embodiments, the individual is human.

In some embodiments, there is provided a method of preventing or treating a skin disease in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for skin conditions. In some embodiments, for each component in the plurality of compositions, the component comprises all of the ingredients listed for the component in Tables 3-9 indicated for skin conditions. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the skin disease is selected from the group consisting of acne, allergies, atopic dermatitis, alopecia, rosacea, eczema and psoriasis. In some embodiments, the individual has one or more risk factors associated with the skin disease. In some embodiments, the individual has had one or more previous occurrences of the skin disease. In some embodiments, the individual is human.

In some embodiments, there is provided a method of preventing or treating a rheumatic disorder in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for joint conditions. In some embodiments, for each component in the composition, the component comprises all of the ingredients listed for the component in Tables 3-9 indicated for joint conditions. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the rheumatic disorder is selected from the group consisting of pain, slow exercise recovery, and arthritis (such as rheumatoid arthritis or osteoarthritis). In some embodiments, the individual has one or more risk factors associated with the rheumatic disorder. In some embodiments, the individual has had one or more previous occurrences of the rheumatic disorder. In some embodiments, the individual is human.

In some embodiments, there is provided a method of preventing or treating a condition resulting from inflammation in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the condition resulting from inflammation is selected from the group consisting of food allergies and sinus allergies. In some embodiments, the individual has one or more risk factors associated with the condition. In some embodiments, the individual has had one or more previous occurrences of the condition. In some embodiments, the individual is human.

In some embodiments, there is provided a method of preventing or treating a pediatric disorder in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the pediatric disorder is selected from the group consisting of allergies, asthma, and GI conditions. In some embodiments, the individual has one or more risk factors associated with the pediatric disorder. In some embodiments, the individual has had one or more previous occurrences of the pediatric disorder. In some embodiments, the individual is human.

In some embodiments, there is provided a method of preventing or treating a food allergy, food sensitivity, or resultant condition in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the food allergy, food sensitivity, or resultant condition is selected from the group consisting of celiac disease, gluten sensitivity, Meniere's disease and osteoarthritis. In some embodiments, the individual is human.

In some embodiments, there is provided a method of preventing or treating an adrenal dysfunction in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for adrenal conditions. In some embodiments, for each component in the composition, the component comprises all of the ingredients listed for the component in Tables 3-9 indicated for adrenal conditions. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the adrenal dysfunction is selected from the group consisting of fatigue and Addison's disease. In some embodiments, the individual is human.

In some embodiments, there is provided a method of preventing or treating a condition associated with GI surgery in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, for each component in the composition, the component comprises at least one ingredient listed for the component in Tables 3-9 indicated for GI conditions. In some embodiments, for each component in the composition, the component comprises all of the ingredients listed for the component in Tables 3-9 indicated for GI conditions. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the individual is human.

In some embodiments, there is provided a method of preventing or treating a condition typically treated with over the counter (OTC) medications or nutritional supplements in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the condition typically treated with over the counter (OTC) medications or nutritional supplements condition is selected from the group consisting of constipation, food and chemical sensitivities, gas/bloating, heartburn/GERD, upset stomach, leaky gut syndrome, food poisoning, diarrhea, traveler's sickness, mild acne, hay fever, allergies, colds and flu, recurring headache, halitosis, weight loss, minor joint pain and stiffness. In some embodiments, the individual is human.

In some embodiments, there is provided a method of preventing or treating an oral disease in an individual comprising administering (such as orally administering) to the individual an effective amount of a composition comprising a probiotic component, colostrum, a protein component and a detoxification component. In some embodiments of the method, the composition further comprises one or more components selected from the group consisting of a prebiotic component, a tissue constituent component, a symptomatic relief component, a cellular bioenergetics component, a tissue healing component and an enzyme component. In some embodiments, the components present in the composition are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the oral disease is selected from the group consisting of halitosis, dry mouth, gingivitis and periodontitis. In some embodiments, the individual has one or more risk factors associated with the oral disease. In some embodiments, the individual has had one or more previous occurrences of the oral disease. In some embodiments, the individual is human.

### Combination Therapy

In some embodiments, there is provided a method of preventing or treating inflammation of a tissue of an individual, comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of preventing or repairing damage (such as tissue damage) in an individual, comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the damage (such as tissue damage) is associated with a disturbance in the integrity of one or more tissue barriers in the individual. In some embodiments, the damage (such as tissue damage) is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the damage (such as tissue damage) is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the damage (such as tissue damage) is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of mitigating leakage of a tissue barrier and/or a mucosal membrane adjacent to or on a tissue of an individual, comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the composition is provided in a dosage form selected from the group consisting of capsules, tablets, soft gels, powder, mist, drops, topical cream, ointment, salve, enema, vaginal or anal suppository, and liquid solution. In some embodiments, the composition is solid. In some embodiments, the composition is a liquid, gel or cream. In some embodiments, where the composition has a high moisture content (such as for a liquid, gel or cream), any microorganism ingredients are added to the composition no more than about 24 hours (such as no more than about 24, 20, 16, 12, 8, 4 or 2 hours) prior to administration. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of reducing the risk of a disease or condition in an individual, comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the disease or condition is associated with a disturbance in the integrity of one or more tissue barriers and/or mucosal membranes adjacent to or on one or more tissues of the individual. In some embodiments, the disease or condition is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the disease or condition is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the disease or condition is associated with a disturbance in the integrity of one or more tissue barriers and/or mucosal membranes adjacent to or on one or more tissues of the individual that provide protection from the external environment and disturbance in the integrity of one or more tissue barriers and/or mucosal membranes adjacent to or on one or more tissues of the individual that provide protection from the internal environment. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, where the plurality of compositions comprises chelation therapy, the chelation therapy is administered after administration of the other compositions in the plurality of compositions. In some embodiments, the individual has one or more risk factors associated with the disease or condition. In some embodiments, the individual has had one or more previous occurrences of the disease or condition. In some embodiments, the individual is human.

In some embodiments, there is provided a method of reducing a side effect associated with the administration of an antibiotic in an individual, comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the side effect associated with the administration of an antibiotic is associated with a disturbance in the integrity of one or more tissue barriers and/or mucosal membranes adjacent to or on one or more tissues of the individual. In some embodiments, the side effect associated with the administration of an antibiotic is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the side effect associated with the administration of an antibiotic is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the side effect associated with the administration of an antibiotic is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of reducing an adverse effect associated with the exposure of an individual to an environmental contaminant or non-natural substance, comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the adverse effect associated with the exposure of an individual to an environmental contaminant or non-natural substance is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of reducing an adverse effect associated with abnormal levels of a naturally occurring substance in an individual, comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the adverse effect associated with abnormal levels of a naturally occurring substance is associated with a disturbance in the integrity of one or more tissue barriers and/or mucosal membranes adjacent to or on one or more tissues of the individual. In some embodiments, the adverse effect associated with abnormal levels of a naturally occurring substance is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the adverse effect associated with abnormal levels of a naturally occurring substance is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the adverse effect associated with abnormal levels of a naturally occurring substance is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the naturally occurring substance is a substance produced by the individual. In some embodiments, the naturally occurring substance is present in the individual in abnormal levels systemically and/or locally. For example, in some embodiments, the naturally occurring substance is present in the individual's blood at a level higher than the normal level found in the blood of a healthy individual, or the naturally occurring substance is present in a particular tissue or organ of the individual at a level higher than the normal level found in the tissue or organ, respectively, of a healthy individual. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of simultaneously treating comorbid conditions in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the comorbid conditions are associated with a disturbance in the integrity of one or more tissue barriers and/or mucosal membranes adjacent to or on one or more tissues of the individual. In some embodiments, the comorbid conditions are associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the comorbid conditions are associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the comorbid conditions are associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of simultaneously enhancing, protecting or repairing the integrity of multiple tissue barriers located in different regions in the body of an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of enhancing, protecting or repairing the integrity of the blood-brain barrier in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of preventing or treating a disease associated with brain or neurological dysfunction in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the disease associated with brain or neurological dysfunction is associated with a disturbance in the integrity of one or more tissue barriers and/or mucosal membranes adjacent to or on one or more tissues of the individual. In some embodiments, the disease associated with brain or neurological dysfunction is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the disease associated with brain or neurological dysfunction is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the disease associated with brain or neurological dysfunction is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual has one or more risk factors associated with the disease. In some embodiments, the individual has had one or more previous occurrences of the disease. In some embodiments, the individual is human.

In some embodiments, there is provided a method of reducing the risk of one or more diseases associated with inflammation in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the diseases associated with inflammation are associated with a disturbance in the integrity of one or more tissue barriers and/or mucosal membranes adjacent to or on one or more tissues of the individual. In some embodiments, the diseases associated with inflammation are associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the diseases associated with inflammation are associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the diseases associated with inflammation are associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the diseases associated with inflammation are selected from the group consisting of cardiovascular disease, food allergies, and sinus allergies. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual has one or more risk factors associated with the disease. In some embodiments, the individual has had one or more previous occurrences of the disease. In some embodiments, the individual is human.

In some embodiments, there is provided a method of preventing or treating diseases associated with exposure to one or more contaminants in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the diseases associated with exposure to one or more contaminants are associated with a disturbance in the integrity of one or more tissue barriers and/or mucosal membranes adjacent to or on one or more tissues of the individual. In some embodiments, the diseases associated with exposure to one or more contaminants are associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the diseases associated with exposure to one or more contaminants are associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the diseases associated with exposure to one or more contaminants are associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the contaminants are selected from the group consisting of chlorine, herbicides, pesticides, antibiotics in foods and drinking or bathing water, hydrocarbons, tar, nicotine, smoke, and heavy metals. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of protecting a tissue after surgery and promoting post-surgical healing of the tissue in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the tissue is GI tissue. In some embodiments, the surgery is GI surgery. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of preventing or treating a disease or condition in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the disease or condition is associated with a disturbance in the integrity of a tissue barrier and/or mucosal membrane adjacent to or on a tissue of the individual. In some embodiments, the disease or condition is associated with a disturbance in the integrity of one or more tissue barriers and/or mucosal membranes adjacent to or on one or more tissues of the individual that provide protection from the external environment, such as those associated with GI, sinus, oral/periodontal, pulmonary, esophageal, vaginal and dermal tissues. In some embodiments, the disease or condition is associated with a disturbance in the integrity of one or more tissue barriers and/or mucosal membranes adjacent to or on one or more tissues of the individual that provide protection from the internal environment, such as those associated with the vascular, hepatic, renal, and urinary tissues. In some embodiments, the disease or condition is associated with a disturbance in the integrity of one or more tissue barriers and/or mucosal membranes adjacent to or on one or more tissues of the individual that provide protection from the external environment and a disturbance in the integrity of one or more tissue barriers and/or mucosal membranes adjacent to or on one or more tissues of the individual that provide protection from the internal environment. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, where the plurality of compositions comprises chelation therapy, the chelation therapy is administered after administration of the other compositions in the plurality of compositions. In some embodiments, the individual has one or more risk factors associated with the disease or condition. In some embodiments, the individual has had one or more previous occurrences of the disease or condition. In some embodiments, the individual is human.

In some embodiments, there is provided a method of preventing or treating a GI condition in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for GI conditions. In some embodiments, for each component in the plurality of compositions, the component comprises all of the ingredients listed for the component in Tables 3-9 indicated for GI conditions. In some embodiments, for each component in the plurality of compositions, the component comprises at least one ingredient listed for the component in Tables 3-9 indicated for diarrhea. In some embodiments, for each component in the plurality of compositions, the component comprises all of the ingredients listed for the component in Tables 3-9 indicated for diarrhea. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the GI condition is selected from the group consisting of irritable bowel syndrome (IBS), inflammatory bowel disease (IBD), colitis, ulcerative colitis, Crohn's disease, diverticulitis, acid reflux, heartburn, gastroesophageal reflux disease (GERD), pouchitis and post-surgical healing. In some embodiments, the individual has one or more risk factors associated with the condition. In some embodiments, the individual has had one or more previous occurrences of the condition. In some embodiments, the individual is human.

In some embodiments, there is provided a method of preventing or treating an autoimmune disease in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the autoimmune disease is selected from the group consisting of acute disseminated encephalomyelitis (ADEM), Addison's disease, allergic granulomatosis and angiitis, alopecia or alopecia areata (AA), ankylosing spondylitis, autoimmune chronic active hepatitis (CAH), autoimmune hemolytic anemia, autoimmune pancreatitis (AIP), autoimmune retinopathy (AR), autoimmune thrombocytopenic purpura, autoimmune neutropenia, autoimmune inner ear disease (AIED), antiphospholipid syndrome (APS), autoimmune lymphoproliferative syndrome (ALPS), Behcet's syndrome, bullus pemphigoid, celiac disease, Cogan's syndrome, Churg-Strauss syndrome (CSS), chronic bullous disease of childhood, chronic inflammatory demyelinating polyradiculoneuropathy (CIDP), cictricial pemphigoid (CP), central nervous system vasculitis, Crohn's disease, cryoglobulinemia, dermatitis herpetiformis (DH), dermatomyositis, discoid lupus erythematosus, encephalomyelitis, epidermolysis bullosa acquisita (EBA), erythema nodosum, Evans syndrome, fibromyalgia, giant cell arteritis, graft-versus-host disease, Graves' disease, Gullain-Barre syndrome, Hanot syndrome, Hashimoto's thyroiditis, hypersensitivity vasculitis (HV), inflammatory bowel disease, insulin-dependent diabetes mellitus (type I diabetes), isolated vasculitis of the central nervous system, Isaacs' syndrome, Kawasaki disease (KD), Lambert-Eaton myasthenic syndrome (LEMS), linear IgA disease, lupus, Meniere's disease, microscopic polyangiitis, mixed connective tissue disease or MCTD, monoclonal gammopathy, myasthenia gravis, multiple sclerosis, multifocal motor neuropathy, neuromyotonia, neutropenia, oophoritis, opsoclonus-myoclonus syndrome, orchitis, paraneoplastic neurologic disorders, pemphigus vulgaris, pemphigus follaceus (PF), pemphigoid gestationis (PG), pernicious anemia, paraneoplastic pemphigus (PNP), polyangiitis, polyarteritis nodosa (PAN), polymyositis, polymyalgia rheumatica, primary biliary cirrhosis (PBC), Primary sclerosing cholangitis (PSC), Psoriasis, Raynaud's phenomenon, recoverin-associated retinopathy (RAR), reactive arthritis, retinopathy, rheumatoid arthritis (RA), sarcoidosis, sclerosing cholangitis see primary sclerosing cholangitis, Sjogren's syndrome, systemic necrotizing vascolitides, stiff man syndrome, systemic lupus erythematosus, systemic sclerosis (scleroderma), temporal arteritis, Takayasu's arteritis, thromboangiitis obliterans, thyroiditis with hypothyroidism, thyroiditis with hyperthyroidism, type I autoimmune polyglandular syndrome (PAS), type II autoimmune polyglandular syndrome, ulcerative colitis, vasculitis and Wegener's granulomatosis. In some embodiments, the individual has one or more risk factors associated with the autoimmune disease. In some embodiments, the individual has had one or more previous occurrences of the autoimmune disease. In some embodiments, the individual is human.

In some embodiments, there is provided a method of preventing or treating a cardiovascular disorder in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for cardiovascular conditions. In some embodiments, for each component in the plurality of compositions, the component comprises all of the ingredients listed for the component in Tables 3-9 indicated for cardiovascular conditions. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the cardiovascular disorder is high blood pressure. In some embodiments, the individual has one or more risk factors associated with the cardiovascular disorder. In some embodiments, the individual has had one or more previous occurrences of the cardiovascular disorder. In some embodiments, the individual is human.

In some embodiments, there is provided a method of preventing or treating a liver disorder in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for liver conditions. In some embodiments, for each component in the plurality of compositions, the component comprises all of the ingredients listed for the component in Tables 3-9 indicated for liver conditions. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the liver disorder is selected from the group consisting of hepatitis, fibrosis, non-alcoholic fatty liver disease (NAFLD) and cirrhosis. In some embodiments, the individual has one or more risk factors associated with the liver disorder. In some embodiments, the individual has had one or more previous occurrences of the liver disorder. In some embodiments, the individual is human.

In some embodiments, there is provided a method of preventing or treating a respiratory disorder in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for respiratory conditions. In some embodiments, for each component in the plurality of compositions, the component comprises all of the ingredients listed for the component in Tables 3-9 indicated for respiratory conditions. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the respiratory disorder is selected from the group consisting of asthma, allergies, hay fever, sinusitis, chronic obstructive pulmonary disease (COPD) and emphysema. In some embodiments, the individual has one or more risk factors associated with the respiratory disorder. In some embodiments, the individual has had one or more previous occurrences of the respiratory disorder. In some embodiments, the individual is human.

In some embodiments, there is provided a method of preventing or treating a rheumatic disorder in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for joint conditions. In some embodiments, for each component in the plurality of compositions, the component comprises all of the ingredients listed for the component in Tables 3-9 indicated for joint conditions. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the rheumatic disorder is selected from the group consisting of pain, slow exercise recovery, and arthritis (such as rheumatoid arthritis or osteoarthritis). In some embodiments, the individual has one or more risk factors associated with the rheumatic disorder. In some embodiments, the individual has had one or more previous occurrences of the rheumatic disorder. In some embodiments, the individual is human.

In some embodiments, there is provided a method of preventing or treating a condition resulting from inflammation in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the condition resulting from inflammation is selected from the group consisting of food allergies and sinus allergies. In some embodiments, the individual has one or more risk factors associated with the condition. In some embodiments, the individual has had one or more previous occurrences of the condition. In some embodiments, the individual is human.

In some embodiments, there is provided a method of preventing or treating a pediatric disorder in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the pediatric disorder is selected from the group consisting of allergies, asthma, and GI conditions. In some embodiments, the individual has one or more risk factors associated with the pediatric disorder. In some embodiments, the individual has had one or more previous occurrences of the pediatric disorder. In some embodiments, the individual is human.

In some embodiments, there is provided a method of preventing or treating a food allergy, food sensitivity, or resultant condition in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the food allergy, food sensitivity, or resultant condition is selected from the group consisting of celiac disease, gluten sensitivity, Meniere's disease and osteoarthritis. In some embodiments, the individual is human.

In some embodiments, there is provided a method of preventing or treating adrenal dysfunction in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for adrenal conditions. In some embodiments, for each component in the plurality of compositions, the component comprises all of the ingredients listed for the component in Tables 3-9 indicated for adrenal conditions. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the adrenal dysfunction is selected from the group consisting of fatigue and Addison's disease. In some embodiments, the individual has one or more risk factors associated with the adrenal dysfunction. In some embodiments, the individual has had one or more previous occurrences of the adrenal dysfunction. In some embodiments, the individual is human.

In some embodiments, there is provided a method of preventing or treating a condition associated with GI surgery in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, for each component in the plurality of compositions, the component comprises at least one ingredient listed for the component in Tables 3-9 indicated for GI conditions. In some embodiments, for each component in the plurality of compositions, the component comprises all of the ingredients listed for the component in Tables 3-9 indicated for GI conditions. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the individual is human.

In some embodiments, there is provided a method of preventing or treating a condition typically treated with over the counter (OTC) medications or nutritional supplements in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the condition typically treated with over the counter (OTC) medications or nutritional supplements condition is selected from the group consisting of constipation, food and chemical sensitivities, gas/bloating, heartburn/GERD, upset stomach, leaky gut syndrome, food poisoning, diarrhea, traveler's sickness, mild acne, hay fever, allergies, colds and flu, recurring headache, halitosis, weight loss, minor joint pain and stiffness. In some embodiments, the individual is human.

In some embodiments, there is provided a method of preventing or treating an oral disease in an individual comprising administering (such as orally administering) to the individual an effective amount of a plurality of compositions comprising a probiotic component, colostrum, a protein component and a detoxification component. In some embodiments of the method, the plurality of compositions further comprises one or more components selected from the group consisting of a prebiotic component, a tissue constituent component, a symptomatic relief component, a cellular bioenergetics component, a tissue healing component and an enzyme component. In some embodiments, the components present in the plurality of compositions are provided according to any of the embodiments described above for compositions of the present invention. In some embodiments, the dosage forms of the compositions in the plurality of compositions are selected from the group consisting of capsules, tablets, soft gels, powder, mist, drops, topical cream, ointment, salve, enema, vaginal or anal suppository, and liquid solution. In some embodiments, at least one composition in the plurality of compositions is a solid. In some embodiments, all the compositions in the plurality of compositions are solid. In some embodiments, at least one composition in the plurality of compositions is any of a liquid, gel or cream. In some embodiments, all the compositions in the plurality of compositions are any of a liquid, gel or cream. In some embodiments, for each composition in the plurality of compositions that has a high moisture content (such as for a liquid, gel or cream), any microorganism ingredients are added to the composition no more than about 24 hours (such as no more than about 24, 20, 16, 12, 8, 4 or 2 hours) prior to administration of the composition. In some embodiments, the plurality of compositions are administered concurrently. In some embodiments, the plurality of compositions are administered sequentially. In some embodiments, at least some of the plurality of compositions are administered sequentially. In some embodiments, the oral disease is selected from the group consisting of halitosis, dry mouth, gingivitis and periodontitis. In some embodiments, the individual has one or more risk factors associated with the oral disease. In some embodiments, the individual has had one or more previous occurrences of the oral disease. In some embodiments, the individual is human.

In some embodiments of the methods presented herein, a plurality of compositions comprises compositions of different dosage forms, such as, but not limited to, capsules, tablets, soft gels, powder, mist, drops, topical cream, ointment, salve, enema, vaginal or anal suppository and liquid solution. In some embodiments, all of the compositions of a plurality of compositions have different dosage forms. In some embodiments, at least two (such as at least any of 2, 3, 4 or 5) of the compositions of a plurality of compositions have the same dosage form. In some embodiments, all of the compositions of a plurality of compositions have the same dosage form. In some embodiments, the number of compositions in the plurality of compositions, and the dosage form of each, is determined based on the dosage form requirements of the ingredients selected to be included in the plurality of compositions according to the condition to be treated and/or the tissue associated with the affected tissue barrier. Thus, for example, where the condition is psoriasis, and the tissue associated with the disturbed tissue barrier is the skin, the plurality of compositions may comprise two compositions of different dosage forms, the first being a tablet composition and the second being a topical cream composition. Thus in some embodiments, each composition of a plurality of compositions comprises ingredients having compatible dosage form requirements, the number of compositions in the plurality of compositions is at least the number of different dosage forms required by the ingredients included in the plurality of compositions, and the dosage form of each composition is a dosage form suitable for all of the ingredients included in that particular composition.

In some embodiments of the methods presented herein, the number of compositions in a plurality of compositions is determined based on the dosing schedule requirements of the ingredients selected to be included in the plurality of compositions according to the condition to be treated and/or the tissue associated with the affected tissue barrier. Thus, for example, where some ingredients are selected to be dosed according to a first schedule of 3 times a day, and the rest are selected to be dosed according to a second schedule of 1 time a day, the plurality of compositions may comprise two compositions, the first composition comprising all the ingredients to be dosed according to the first schedule, and the second composition comprising all the ingredients to be dosed according to the second schedule. Thus in some embodiments, each composition of a plurality of compositions comprises ingredients having compatible dosing schedule requirements and the number of compositions in the plurality of compositions is at least the number of different dosing schedules required by the ingredients included in the plurality of compositions. It is further appreciated that the number of compositions in the plurality of compositions will be affected by the interaction of dosage form requirements and dosing schedule requirements of the included ingredients as described above. Thus in some embodiments, each composition of a plurality of compositions comprises ingredients having compatible dosage form and dosing schedule requirements and the number of compositions in the plurality of compositions is at least the larger of the number of different dosage forms and the number of different dosing schedules required by the ingredients included in the plurality of compositions.

In some embodiments, according to any of the methods of preventing or treating a disease or condition in an individual described above, the method further comprises determining affected tissue(s) and/or a manifestation stage for the disease or condition and selecting the components of the composition or plurality of compositions based on the determination of the affected tissue(s) and/or manifestation stage. In some embodiments, the manifestation stage of the disease or condition is determined according to Table 10. In some embodiments, the amount of each ingredient in the composition or plurality of compositions is about 0.1 to about 100 times (including for example about 0.25 to about 20 times, about 0.5 to about 10 times, or about any of 0.25×, 0.5×. 1×, 5×, 10×, 15×, or 20×) the specific amount listed for the ingredient in Tables 3-9 for the determined manifestation stage. In some embodiments, the amount of each ingredient in the composition or plurality of compositions is about the specific amount listed for the ingredient in Tables 3-9 for the determined manifestation stage. In some embodiments, the amounts of each ingredient in the composition or plurality of compositions are provided at the same relative weight ratios as the weight ratios calculated based on the specific amounts for the ingredients listed in Tables 3-9 for the determined manifestation stage. In some embodiments, for each component in the composition or plurality of compositions, the component comprises at least one of the items listed for the component in Tables 3-9 indicated for the determined affected tissue(s). In some embodiments, for each component in the composition or plurality of compositions, the component comprises all of the items listed for the component in Tables 3-9 indicated for the determined affected tissue(s).

TABLE 10
Stages and manifestations of complications

| | | | | | |
|---|---|---|---|---|---|
| Complication | | | | | |
| area | Stage 1 | Stage 2 | Stage 3 | Stage 4 | Leaky gut |

| | | | | |
|---|---|---|---|---|
| GI | Recurring diarrhea, | Colitis/pouchitis, post- | Ulcerative colitis, | Crohn's |
| | constipation, IBS, J-pouch | surgical healing, food | celiac disease | disease, |
| | maintenance, food | allergy | | diverticulitis |
| | sensitivity | | | |
| Non-adjacent | <1 year | 1-5 years | >5 years | >10 years |
| autoimmune | | | | |
| disease | | | | |
| Skin/hair | Acne/allergy | Eczema, alopecia | Psoriasis | |
| Brain | Recurring headache | | | |
| Neurological | Chronic fatigue | | | |
| Liver | NAFLD | Fibrosis | Hepatitis | Cirrhosis |
| Respiratory | Recurring colds & flu, | Asthma | Smoker/COPD | Emphysema |
| | allergy, hay fever | | | |
| Poisoning | Food | Mercury | Antimicrobials | |
| Joints | | Rheumatoid arthritis | Osteoarthritis | |
| Adrenal | Adrenal fatigue | Adrenal failure | Addison's | |
| Weight | | Syndrome X | Type II diabetes, | |
| | | | NAFLD | |
| Inflammation | Stress | Local inflammation | Chronic | |
| | | | inflammation | |
| Hearing | Tinnitus | | Hearing loss | |
| Mouth | Bad breath, dry mouth | Gingivitis | Periodontitis | |

In some embodiments of the methods presented herein, the method further comprises a) first administering to the individual the selected composition or plurality of compositions comprising ingredients at their maintenance doses; and b) incrementally increasing the doses of the ingredients over a transition period to reach the doses indicated for the ingredients at the manifestation stage determined for the disease or condition. In some embodiments, the doses are incrementally increased by about 10% to about 100% (such as by about any of 15%, 20%, 25%, 30%, 35%, 40%, 45%, 50%, 55%, 60%, 65%, 70%, 75%, 80%, 85%, 90%, 95%) In some embodiments, where an increase in the doses of the ingredients coincides with an adverse reaction in the individual, the doses of the ingredients are not increased until the adverse reaction has stabilized. In some embodiments, the transition period is from about 1 to about 20 (such as about any of 1, 2, 3, 4, 5, 6, 7, 8, 9, 10, 12, 14, 16, 18 or 20) days. In some embodiments, the dose is increased at an interval from about 1 to about 5 (such as about any of 1, 2, 3, 4 or 5) days during the transition period.

In some embodiments of the methods presented herein, the method further comprises a) first administering to the individual the selected composition or plurality of compositions comprising ingredients at the doses indicated for the manifestation stage determined for the disease or condition until the symptoms of the disease or condition have abated; b) incrementally decreasing the dose of any pharmaceutical drugs being taken by the individual, wherein the decrease is halted at recurrence of symptoms, and resumed once symptoms have stabilized; and c) incrementally decreasing the doses of the ingredients of the selected composition or plurality of compositions until reaching their maintenance doses. In some embodiments, the doses of the pharmaceutical drug are decreased by increments from about 0.1 to about 0.5 (such as about any of 0.1, 0.2, 0.3, 0.4 or 0.5) times the original doses. In some embodiments, the doses of the ingredients of the selected composition or plurality of compositions are decreased by increments from about 0.1 to about 0.5 (such as about any of 0.1, 0.2, 0.3, 0.4 or 0.5) times the original doses.

In some embodiments of the methods presented herein, the composition or plurality of compositions is administered to the individual as divided daily doses, wherein the sum of the divided daily doses is about equal to the daily dose. In some embodiments, the composition or plurality of compositions is divided into at least 2 (such as at least any of 2, 3, 4, 5, 6, 7 or 8) daily doses. In some embodiments, the divided daily doses are about equal. In some embodiments, the divided daily doses are not about equal.

In some embodiments of the methods presented herein, any compositions comprising micronutrients administered to the individual orally are taken with a meal. In some embodiments, any compositions comprising core components (probiotic component, colostrum, protein component, and detoxification component) administered to the individual orally are taken either 1 hour before or 1 hour after a meal, with room temperature or colder liquid, such as, but not limited to, water.

In some embodiments of the methods presented herein, where the individual was not breastfed for at least one year, and where an ingredient has a dose range indicated, the manifestation stage dose is chosen to be the higher end of the range.

In some embodiments of the methods presented herein, the composition or plurality of compositions does not include ingredients that promote toxin and/or free radical removal until symptoms of the condition in the individual have stabilized. In some embodiments, the composition or plurality of compositions does not include enzymes that dissolve and/or reduce scabs, callouses and scarring until symptoms of the condition in the individual have stabilized.

In some embodiments of the methods presented herein, where the condition to be treated is a condition of the oral cavity, any composition comprising core components (probiotic component, colostrum, protein component and detoxification component) administered orally is not encapsulated, and is kept in the mouth for a period of time from about 0.5 to about 2 (such as about 1) minutes with agitation before swallowing. In some embodiments, where the condition to be treated is a condition of the oral cavity, any composition comprising core components (probiotic component, colostrum, protein component and detoxification component) administered orally is not encapsulated, and is kept in the mouth for a period of time from about 0.5 to about 2 (such as about 1) minutes without agitation before swallowing. In some embodiments, where the condition to be treated is a condition of the oral cavity, the individual does not eat or drink for a period of time from about 5 to about 30 (such as about any of 10, 15, 20 or 25) minutes after administering an oral composition comprising core components (probiotic component, colostrum, protein component and detoxification component).

In some embodiments of the methods presented herein, where the individual is not an adult, the doses of ingredients in the composition or plurality of compositions are adjusted to be from about 0.5 to about 1.5 (such as about 1) times the ratio of the individual's body weight to the mean body weight of an adult of the same species. Thus, for example, for a non-adult individual having half the body weight of an average adult of the same species, the doses of ingredients in the composition or plurality of compositions are adjusted to be from about 0.25 to about 0.75 (such as about 0.5) times the indicated doses.

In some embodiments of the methods presented herein, where a dose is not taken at the scheduled time, it may be taken at any time up to and including the scheduled time for the next dose. Thus, for example, for a method comprising 3 divided daily doses scheduled to be taken at 8 am, 2 pm and 8 pm, if the Sam dose is not taken at about 8 am, it may be taken at any time after about Sam and up to about 2 pm (such as at about any of 10 am, 11 am, 12 pm, 1 pm or 2 pm).

In some embodiments of the methods presented herein, where the individual has an allergy or sensitivity to an ingredient of the composition or plurality of compositions, the ingredient will not be included in the composition or plurality of compositions. In some embodiments, where the individual has an allergy or sensitivity to an ingredient of the composition or plurality of compositions, an alternative ingredient that the individual is not allergic or sensitive to will be included. Thus, for example, where the individual has an allergy to milk proteins, the composition or plurality of compositions will not include whey protein, and will include non-dairy protein, such as, but not limited to, beef protein, as an alternative.

In some embodiments of the methods presented herein, the method further comprises monitoring the progress of the individual. In some embodiments, the monitoring comprises recording symptoms and any relevant indicators (such as, but not limited to, plasma high-sensitivity CRP, 25(OH)D and visible organ dysbiosis) present in the individual using any method known in the art. In some embodiments, the symptoms comprise symptoms of the condition to be treated. In some embodiments, the symptoms further comprise symptoms unrelated to the condition to be treated. In some embodiments, the monitoring further comprises recording a baseline of the symptoms and any relevant indicators prior to treatment.

In some embodiments of the methods presented herein, the method further comprises removing precipitating factors from the individual. In some embodiments, the precipitating factors comprise antibiotics, chlorine (through ingestion, inhalation or skin absorption), alcohol (through ingestion or skin absorption), antibacterial agents from household goods, acetaminophen, pesticides, fungicides, herbicides, hydrocarbons, phosphoric acid, food contaminants, or a combination thereof. In some embodiments, where the condition is a skin condition, the precipitating factors further comprise cosmetics, sunscreen, rubber, black clothing dye or a combination thereof. In some embodiments, where the condition is a sinus or lung condition, the precipitating factors further comprise cigarette smoke, chlorine mist (such as from shower), regular exposure to chemical fumes or a combination thereof. In some embodiments, where the condition is a cardiovascular condition, the precipitating factors further comprise excess homocysteine. In some embodiments, where the condition is an oral condition, the precipitating factors further comprise antibacterial or alcohol-based mouthwash.

In some embodiments of the methods presented herein, the method further comprises temporarily removing or reducing irritating factors from the individual until the integrity of the tissue barrier is restored. In some embodiments, where the condition is a GI condition, the irritating factors comprise fiber, roughage, fruit, raw vegetables, nuts, seeds, popcorn, corn-containing products, digestive enzymes, hydrochloric acid or a combination thereof.

In some embodiments of the methods presented herein, the method further comprises temporarily removing allergens from the individual. In some embodiments, the allergens comprise oral, topical or airborne allergens.

In some embodiments of the methods presented herein, the method further comprises using a pharmaceutical drug for symptomatic relief. The pharmaceutical drug for symptomatic relief does not comprise immune destroying drugs (such as infliximab) or wide-spectrum antibiotics. In some embodiments, the individual is not undergoing long-term antibiotic administration or administration of drugs with known side effects that include intestinal distress, dry membranes, mitochondrial damage or other symptoms of tissue barrier dysbiosis.

In some embodiments of the methods presented herein, the tissue barrier is adjacent to or on a tissue that includes, but is not limited to, GI tract, nasal tissue, lung, liver, mouth, vagina, urinary tract, bladder, ear, eye, epithelium, fat and muscle

In some embodiments of the methods presented herein, the diseases or conditions to be treated include, but are not limited to: a) GI conditions, b) auto-immune disease, c) leaky membranes and resultant diseases, d) vascular disorders, e) kidney disorders, f) heart and circulatory disorders, g) liver disorders, h) respiratory disorders, i) skin diseases, j) rheumatic disorders, k) oral diseases, l) poisoning by food or heavy metals, m) pediatric disorders, n) food allergies and/or sensitivities and cascading effects, o) side effects of antimicrobials, p) abnormal weight change, q) adrenal dysfunction, r) side effects of pharmaceutical drugs, s) conditions associated with topical absorption of toxic contaminants, t) conditions associated with hospitalization and surgery, and u) conditions typically treated with over the counter (OTC) medications and nutritional supplements.

GI conditions include, but are not limited to, irritable bowel syndrome (IBS), inflammatory bowel disease (IBD), colitis, ulcerative colitis, Crohn's disease, diverticulitis, acid reflux, heartburn, gastroesophageal reflux disease (GERD), pouchitis and post-surgical healing.

Autoimmune diseases include, but are not limited to, acute disseminated encephalomyelitis (ADEM), Addison's disease, allergic granulomatosis and angiitis, alopecia or alopecia areata (AA), ankylosing spondylitis, autoimmune chronic active hepatitis (CAH), autoimmune hemolytic anemia, autoimmune pancreatitis (AIP), autoimmune retinopathy (AR), autoimmune thrombocytopenic purpura, autoimmune neutropenia, autoimmune inner ear disease (AIED), antiphospholipid syndrome (APS), autoimmune lymphoproliferative syndrome (ALPS), Behcet's syndrome, bullus pemphigoid, celiac disease, Cogan's syndrome, Churg-Strauss syndrome (CSS), chronic bullous disease of childhood, chronic inflammatory demyelinating polyradiculoneuropathy (CIDP), cictricial pemphigoid (CP), central nervous system vasculitis, Crohn's disease, cryoglobulinemia, dermatitis herpetiformis (DH), dermatomyositis, discoid lupus erythematosus, encephalomyelitis, epidermolysis bullosa acquisita (EBA), erythema nodosum, Evans syndrome, fibromyalgia, giant cell arteritis, graft-versus-host disease, Graves' disease, Gullain-Barre syndrome, Hanot syndrome, Hashimoto's thyroiditis, hypersensitivity vasculitis (HV), inflammatory bowel disease, insulin-dependent diabetes mellitus (type I diabetes), isolated vasculitis of the central nervous system, Isaacs' syndrome, Kawasaki disease (KD), Lambert-Eaton myasthenic syndrome (LEMS), linear IgA disease, lupus, Meniere's disease, microscopic polyangiitis, mixed connective tissue disease or MCTD, monoclonal gammopathy, myasthenia gravis, multiple sclerosis, multifocal motor neuropathy, neuromyotonia, neutropenia, oophoritis, opsoclonus-myoclonus syndrome, orchitis, paraneoplastic neurologic disorders, pemphigus vulgaris, pemphigus follaceus (PF), pemphigoid gestationis (PG), pernicious anemia, paraneoplastic pemphigus (PNP), polyangiitis, polyarteritis nodosa (PAN), polymyositis, polymyalgia rheumatica, primary biliary cirrhosis (PBC), Primary sclerosing cholangitis (PSC), Psoriasis, Raynaud's phenomenon, recoverin-associated retinopathy (RAR), reactive arthritis, retinopathy, rheumatoid arthritis (RA), sarcoidosis, sclerosing cholangitis see primary sclerosing cholangitis, Sjogren's syndrome, systemic necrotizing vascolitides, stiff man syndrome, systemic lupus erythematosus, systemic sclerosis (scleroderma), temporal arteritis, Takayasu's arteritis, thromboangiitis obliterans, thyroiditis with hypothyroidism, thyroiditis with hyperthyroidism, type I autoimmune polyglandular syndrome (PAS), type II autoimmune polyglandular syndrome, ulcerative colitis, vasculitis and Wegener's granulomatosis.

In some embodiments, the autoimmune disease affects a tissue/organ selected from the group consisting of adrenal gland, intestine, muscle, thyroid gland, skin, kidney, joint, brain, spinal cord, stomach, salivary gland, lacrimal gland, and pancreas.

Vascular disorders include, but are not limited to, high blood pressure and arteriosclerosis.

Liver disorders include, but are not limited to, hepatitis, fibrosis, non-alcoholic fatty liver disease (NAFLD) and cirrhosis.

Respiratory disorders include, but are not limited to, asthma, allergies, hay fever, sinusitis, chronic obstructive pulmonary disease (COPD) and emphysema.

Skin diseases include, but are not limited to, acne, allergies, atopic dermatitis, alopecia, rosacea, eczema and psoriasis.

Rheumatic disorders include, but are not limited to, pain, slow exercise recovery, and arthritis (such as rheumatoid arthritis or osteoarthritis).

Inflammation and resultant conditions include, but are not limited to, food allergies and sinus allergies.

Pediatric disorders include, but are not limited to, allergies, asthma, and GI conditions.

Food allergies and/or sensitivities and cascading effects include, but are not limited to, celiac disease, gluten sensitivity, Meniere's disease and osteoarthritis.

Adrenal dysfunction conditions include, but are not limited to, fatigue and Addison's disease.

Conditions associated with hospitalization and surgery include, but are not limited to, conditions associated with GI surgery.

Conditions typically treated with over the counter (OTC) medications and nutritional supplements include, but are not limited to, constipation, food and chemical sensitivities, gas/bloating, heartburn/GERD, upset stomach, leaky gut syndrome, food poisoning, diarrhea, traveler's sickness, mild acne, hay fever, allergies, colds and flu, recurring headache, halitosis, weight loss, minor joint pain and stiffness.

Oral diseases include, but are not limited to, halitosis, dry mouth, gingivitis and periodontitis.

In some embodiments of the methods presented herein, the individual is a healthy individual. In some embodiments, the individual is a diseased individual (such as an individual having any one or more of the diseases or conditions described herein). In some embodiments, the individual is on dietary restriction on dietary fiber. In some embodiments, the individual is younger than about 60 years old (including for example an individual younger than about 50, 40, 30, 25, 20, 15, or 10 years old). In some embodiments, the individual is older than about 60 years old (including, for example, 70, 80, 90, or 100 years old). In some embodiments, the individual is an infant. In some embodiments, the individual is a child. In some embodiments, the individual is an adult. In some embodiments, the individual is a pregnant female. In some embodiments, the individual is diagnosed with or genetically prone to one or more of the diseases or conditions described herein. In some embodiments, the individual has one or more risk factors associated with one or more diseases or conditions described herein. In some embodiments, the individual has had one or more previous occurrences of one or more diseases or conditions described herein.

In some embodiments of the methods presented herein, the individual is a human.

The composition or plurality of compositions can be administered at any desired frequency. In some embodiments of the methods presented herein, the composition or plurality of compositions is administered once daily, twice daily, thrice daily, or more frequently. In some embodiments, where a plurality of compositions is administered, all of the compositions in the plurality of compositions are administered at the same frequency. In some embodiments, where a plurality of compositions is administered, at least some of the compositions in the plurality of compositions are administered at different frequencies. In some embodiments, where a plurality of compositions is administered, each of the compositions in the plurality of compositions is administered concurrently. In some embodiments, where a plurality of compositions is administered, at least some of the compositions in the plurality of compositions are not administered concurrently. Thus, for example, a tablet composition may be administered 3 times daily, and a topical cream may be administered once daily, wherein the topical cream is administered at the same time as a tablet, or at a different time than any of the tablets.

In some embodiments of the methods presented herein, a composition is administered along with food. In some embodiments, a composition is not administered along with food. In some embodiments, a composition is administered on an empty stomach. In some embodiments, a composition is administered in conjunction with a dietary supplement. In some embodiments, a composition is administered in conjunction with water. In some embodiments, the method comprises administering the composition or plurality of compositions at a therapeutic dose, followed by administering the composition or plurality of compositions at a maintenance dose.

### Prevention

In some embodiments, according to any of the methods described above for preventing or treating a disease or condition, the preventing comprises eliminating the occurrence of the disease or condition in the individual for at least about 6 months (such as at least about any of 7 months, 8 months, 9 months, 10 months, 11 months, 12 months, 13 months, 14 months, 15 months, 16 months, 17 months, 18 months, 2 years, 3 years, 4 years, 5 years, or more, including any ranges between these values) following initiation of the method. In some embodiments, the individual has one or more risk factors associated with the disease or condition. In some embodiments, the individual has had one or more previous occurrences of the disease or condition. In some embodiments, the individual is human.

In some embodiments, according to any of the methods described above for preventing or treating a disease or condition, the preventing comprises reducing the incidence rate of the disease or condition in the individual. In some embodiments, the incidence rate for a post-method period of time is reduced by at least about 20% (such as by at least about any of 25%, 30%, 35%, 40%, 45%, 50%, 55%, 60%, 65%, 70%, 75%, 80%, 85%, 90%, 95%, or more) as compared to the incidence rate for a pre-method period of time. In some embodiments, the post-method period of time ranges between at most about 3 months (such as at most about any of 2.5 months, 2 months, 1.5 months, 1 month, 2 weeks, or less, including any ranges between these values) to at least about 6 months (such as at least about any of 7 months, 8 months, 9 months, 10 months, 11 months, 12 months, 13 months, 14 months, 15 months, 16 months, 17 months, 18 months, 24 months, or more, including any ranges between these values) following initiation of the method. In some embodiments, the pre-method period of time ranges between at least about 6 months (such as at least about any of 7 months, 8 months, 9 months, 10 months, 11 months, 12 months, 13 months, 14 months, 15 months, 16 months, 17 months, 18 months, 2 years, 3 years, 4 years, 5 years, or more, including any ranges between these values) to at most about 1 month (such as at most about any of 3 weeks, 2 weeks, 1 week, 3 days, 1 day, or less, including any ranges between these values) prior to initiation of the method. In some embodiments, the individual has one or more risk factors associated with the disease or condition. In some embodiments, the individual has had one or more previous occurrences of the disease or condition. In some embodiments, the individual is human.

In some embodiments, according to any of the methods described above for preventing or treating a disease or condition, the individual presents one or more markers (such as aberrant protein expression) associated with the disease or condition, and the preventing comprises reducing or eliminating presentation of at least one of the one or more markers. In some embodiments, the presentation of the at least one of the one or more markers is reduced by at least about 20% (such as by at least about any of 25%, 30%, 35%, 40%, 45%, 50%, 55%, 60%, 65%, 70%, 75%, 80%, 85%, 90%, 95%, or more) following an effective period of time after initiation of the method. In some embodiments, the effective period of time is at least about 2 weeks (such as at least about any of 3 weeks, 1 month, 1.5 months, 2 months, 2.5 months, 3 months, 3.5 months, 4 months, 4.5 months, 5 months, 5.5 months, 6 months, 7 months, 8 months, 9 months, 10 months, 11 months, 12 months, or more, including any ranges between these values). In some embodiments, the individual has one or more risk factors associated with the disease or condition. In some embodiments, the individual has had one or more previous occurrences of the disease or condition. In some embodiments, the individual is human.

For example, in some embodiments, according to any of the methods described above for preventing or treating a disease or condition, the individual has aberrant expression of one or more proteins associated with the disease or condition, and the preventing comprises reducing or eliminating the aberrant expression of at least one of the one or more proteins. Aberrant protein expression can be measured, for example, as the difference between the expression of the protein in a tissue or fluid of the individual and a reference value for the expression of the protein in the tissue or fluid, respectively, of a healthy individual. In some embodiments, the aberrant expression of the at least one of the one or more proteins is decreased by at least about 20% (such as by at least about any of 25%, 30%, 35%, 40%, 45%, 50%, 55%, 60%, 65%, 70%, 75%, 80%, 85%, 90%, 95%, or more) following an effective period of time after initiation of the method. In some embodiments, the effective period of time is at least about 2 weeks (such as at least about any of 3 weeks, 1 month, 1.5 months, 2 months, 2.5 months, 3 months, 3.5 months, 4 months, 4.5 months, 5 months, 5.5 months, 6 months, 7 months, 8 months, 9 months, 10 months, 11 months, 12 months, or more, including any ranges between these values). In some embodiments, the individual has one or more risk factors associated with the disease or condition. In some embodiments, the individual has had one or more previous occurrences of the disease or condition. In some embodiments, the individual is human.

In some embodiments, there is provided a method of treating or preventing a disease or condition in an individual by following a protocol as described in any of Examples 2-5.

### Articles of Manufacture and Kits

Also provided herein are articles of manufacture and kits useful for carrying out one or more of the methods described herein. The article of manufacture can comprise a container and a label or package insert on or associated with the container. Suitable containers include, for example, bottles, vials, syringes, etc. The containers may be formed from a variety of materials such as glass or plastic. Generally, the container holds a composition according to any of the embodiments described above which is effective for treating a disease or disorder described herein, and may have a sterile access port (for example the container may be an intravenous solution bag or a vial having a stopper pierceable by a hypodermic injection needle). The label or package insert indicates that the composition is used for treating the particular condition. The label or package insert will further comprise instructions for administering the composition to a patient. Articles of manufacture and kits comprising a plurality of compositions are also contemplated.

Package insert refers to instructions customarily included in commercial packages of therapeutic products that contain information about the indications, usage, dosage, administration, contraindications and/or warnings concerning the use of such therapeutic products.

Kits are also provided that are useful for various purposes, e.g., for treatment of a disease or disorder described herein, optionally in combination with the articles of manufacture. Kits of the invention include one or more containers comprising a composition or plurality of compositions according to any of the embodiments described above (or unit dosage form and/or article of manufacture), and in some embodiments, further comprise instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises one or more of the ingredients listed in tables 3-8. In some embodiments, the kit comprises all of the ingredients listed in tables 3-8. In some embodiments, the ingredients provided with the kit are in a quantity sufficient for any of about 30 to about 90 (such as about any of 30, 40, 50, 60, 70, 80 or 90) days of a normal adult dosing. In some embodiments, the kit further comprises instructions for administering the composition or plurality of compositions in accordance with any of the methods described herein.

### EXAMPLES

The following non-limiting examples further illustrate the compositions and methods of the present invention. Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1. Use of Patient Studies to Determine Efficacy of a Composition or Plurality of Compositions

If desired, a composition or plurality of compositions as described herein can be tested in humans to determine its ability to prevent or treat any of the diseases or conditions described herein. Standard methods can be used.

In one exemplary method, subjects (such as healthy subjects, subjects with one of the diseases or conditions, or subjects at increased risk for developing one of the diseases or conditions) are administered a composition or plurality of compositions according to a method of the present invention. To evaluate the efficacy of the method of treating a disease or condition, subjects suffering from the disease or condition are administered either a composition or plurality of compositions according to the method (experimental group) or a placebo (control group) and are monitored for symptoms and related indicators of the disease or condition prior to and throughout the treatment. The extent to which symptoms resolve and related indicators return to normal levels is compared between the experimental and control groups.

To evaluate the efficacy of the method of preventing a disease or condition, healthy subjects and/or subjects at increased risk for developing the disease or condition are administered either a composition or plurality of compositions according to the method (experimental group) or a placebo (control group) and are monitored for symptoms and related indicators of the disease or condition prior to and throughout the treatment. The frequency of occurrence of the disease or condition is compared between the experimental and control groups.

### Example 2. Protocol for Treatment of Gastrointestinal Dysfunction

### Diagnosis

The following table categorizes disease diagnosis and protocol dosing from lowest diagnosable manifestation to most damaging. The Leaky Gut column on the far right does not follow this rule. For difficult cases, the dosing can be doubled. To save costs on maintenance, dosing may be halved, but there is a risk that the condition will reappear.

TABLE 11
Manifestation stages of GI conditions associated with tissue barrier dysfunction

| | Stage 1 | Stage 2 | Stage 3 | Stage 4 | Leaky gut |
|---|---|---|---|---|---|
| GI | IBS, J-pouch | colitis, pouchitis, | ulcerative | Crohn's | intestinal |
| dysfunction | maintenance, food | post-surgical | colitis, celiac | disease, | hyper- |
| | sensitivities | healing, food | disease | diverticulitis | permeability |
| | | allergies | | | |

### Specific Treatments

Patients diagnosed with gastrointestinal dysfunction are administered a treatment regimen that includes a composition or plurality of compositions comprising one or more of the items listed in Tables 12-16 (presented as adult daily dosage, which is best taken as a divided dose, and adjusted for weight, such as for children).

TABLE 12
Mucosal milieu treatment - oral for intestinal milieu

| | Maintenance | | Stage 1 | | Stage 2 | | Stage 3 | | Stage 4 | | | |
|---|---|---|---|---|---|---|---|---|---|---|---|---|
| Ingredient | Dose | | Dose | | Dose | | Dose | | Dose | | Leaky gut | |
| Probiotic blend^{1,2} | 30-60B³ | | 60-120B | | 120-240B | | 180-360B | | 180-720B | | 120-360B | |
| Colostrum⁴ | 1-2 | g | 2-4 | g | 4-8 | g | 6-12 | g | 6-24 | g | 4-12 | g |
| Prebiotics | 250-500 | mg | 0.5-1 | g | 1-2 | g | 1.5-3 | g | 1.5-6 | g | 1-3 | g |
| Whey Protein | 5-10 | g | 10-20 | g | 20-40 | g | 30-60 | g | 30-120 | g | 20-60 | g |
| Beef Protein⁵ | 5-10 | g | 10-20 | g | 20-40 | g | 30-60 | g | 30-120 | g | 20-60 | g |

| | | | | | | |
|---|---|---|---|---|---|---|
| Bacillus coagulans⁶ | 5B | 10B | 20B | 20B | 20B | 20B |

¹A complex blend of acid-resistant probiotic strains. Refrigeration is suggested to maintain potency.
²Our formulation is loaded with >4 times the label claim of CFU (live colony forming units) (i.e.: 110 billion CFU at time of bottling is guaranteed to deliver 25 billion CFU at expiration date when stored under refrigeration). So, the actual CFU at time of use for the product labeled 25 billion CFU may vary from 100 billion to 25 billion. Stability testing on this product indicates the formula drops to 25 billion 14 months after bottling when stored at ambient room temperature (70 degrees F.).
³B = billion CFU (Colony Forming Units) (live organisms)
⁴High in immunoglobulin content, high in protein-rich polypeptides (PRP), low in milk proteins.
⁵Alternative to Whey Protein for patients who are allergic/sensitive to milk/casein products.
⁶For use in conjunction with probiotic blend when environmental factors make the preferred probiotic blend less effective: in conjunction with antibiotic therapy or when traveling and refrigeration not available.

TABLE 13
Organic Constituents of Tissue - oral

| | Maintenance | | Stage 1 | | Stage 2 | | Stage 3 | | Stage 4 | | | |
|---|---|---|---|---|---|---|---|---|---|---|---|---|
| Ingredient | Dose | | Dose | | Dose | | Dose | | Dose | | Leaky gut | |
| Krill oil¹ | 0.5 | g | 0.5-1 | g | 1-2 | g | 2-4 | g | 4-8 | g | 1-3 | g |
| Fish oil² | 3 | g | 3-5 | g | 5-10 | g | 10-20 | g | 20-40 | g | 5-15 | g |
| Phospholipids^{2,3} | 150 | mg | 150-300 | mg | 300-600 | mg | 0.6-1.2 | g | 1.2-2.4 | g | 0.3-0.9 | g |
| L-glutamine | 0.5 | g | 0.75 | g | 1.5 | g | 3 | g | 4.5 | g | 1.5-3 | g |
| N-acetyl glucosamine | 0.25 | g | 0.5 | g | 1 | g | 2 | g | 3 | g | 1-2 | g |
| Methylsulfonylmethane (MSM) | 25 | mg | 50 | mg | 100 | mg | 200 | mg | 300 | mg | 100-200 | mg |
| Zinc Carnosine | 25 | mg | 50 | mg | 75 | mg | 150 | mg | 225 | mg | 75-150 | mg |
| Micronutrient Therapy⁴ | | | | | | | | | | | | |

| | |
|---|---|
| Vitamin D3 | typically 5,000 to |
| | 10,000 IU/day⁵ |

¹Containing EPA, DHA, and other PUFAs (polyunsaturated fatty acids) bound to 2 or more of 69 different phospholipid molecules, plus the antioxidant astaxanthin.
²Alternative to krill oil
³As phospholipid complex consisting of Phosphatidyl Serine, Phosphatidyl Choline, Phosphatidyl Ethanolamine, Phosphatidyl Inositol, Phosphatidic Acid, and Acetylated Phosphatidyl Ethanolamine.
⁴Sophisticated multivitamin such as Rejuvenation Science Maximum Vitality taken at standard dose with food.
⁵Dosed to achieve 80-100 ng/ml 25(OH)D plasma level.

TABLE 14
Symptomatic Relief

| | Maintenance | | Stage 1 | | Stage 2 | | Stage 3 | | Stage 4 | | | |
|---|---|---|---|---|---|---|---|---|---|---|---|---|
| Ingredient | Dose | | Dose | | Dose | | Dose | | Dose | | Leaky gut | |
| Citrus Pectin | 0.25 | g | 0.5 | g | 1 | g | 2 | g | 3 | g | 1-2 | g |
| Deglcyrrhizinated Licorice (DGL) | 125 | mg | 250 | mg | 400 | mg | 800 | mg | 1.2 | g | 400-800 | mg |
| Aloe Vera Extract (Aloe barbadensis) (leaf) | 100 | mg | 150 | mg | 300 | mg | 600 | mg | 900 | mg | 300-600 | mg |
| Slippery Elm (Ulmus fulva)(bark) | 75 | mg | 100 | mg | 200 | mg | 400 | mg | 600 | mg | 200-400 | mg |
| Mucin | 75 | mg | 100 | mg | 200 | mg | 400 | mg | 600 | mg | 200-400 | mg |
| Marshmallow (Althaea officinalis)(root) | 25 | mg | 50 | mg | 100 | mg | 200 | mg | 300 | mg | 100-200 | mg |
| Chamomile (Matricaria chamomilla)(flower) | 25 | mg | 50 | mg | 100 | mg | 200 | mg | 300 | mg | 100-200 | mg |
| Okra Extract (Abelmoschus esculentus)(fruit) | 25 | mg | 50 | mg | 100 | mg | 200 | mg | 300 | mg | 100-200 | mg |
| Cat's Claw (Uncaria tomentosa)(bark) | 25 | mg | 50 | mg | 100 | mg | 200 | mg | 300 | mg | 100-200 | mg |
| Quercetin | 25 | mg | 50 | mg | 100 | mg | 200 | mg | 300 | mg | 100-200 | mg |
| Sodium Butyrate¹ | 25 | mg | 50 | mg | 100 | mg | 200 | mg | 300 | mg | 100-200 | mg |
| Saccharomyces Boulardii² | | | | | | | | | | | | |

¹Alternatively calcium butyrate or magnesium butyrate.
²As needed for diarrhea. Start with 5B CFU and titrate up until symptoms abate, then taper down as treatment progresses and symptoms subside.

TABLE 15
Cellular Bioenergetics - oral

| | Maintenance | | Stage 1 | | Stage 2 | | Stage 3 | | Stage 4 | | | |
|---|---|---|---|---|---|---|---|---|---|---|---|---|
| Ingredient | Dose | | Dose | | Dose | | Dose | | Dose | | Leaky gut | |
| CoQ10 (ubiquinol) | 50 | mg | 100 | mg | 100-200 | mg | 200-400 | mg | 300-600 | mg | | |
| CoQ10 (ubiquinone)¹ | 400-500 | mg | 0.8-1 | g | 0.8-2 | g | 1.6-4 | g | 2.4-6 | g | | |
| L-Carnitine² | 500 | mg | 0.5-1 | g | 1-2 | g | 2-2.5 | g | 2.5-3.5 | g | 1-2.5 | g |
| PQQ² | — | | 10 | mg | 20 | mg | 30 | mg | 40 | mg | 20-30 | mg |
| D-ribose² | 5 | g | 5-10 | g | 10-15 | g | 10-15 | g | 15 | g | 10-15 | g |
| Alpha-Lipoic Acid² | 50 | mg | 100 | mg | 200 | mg | 400 | mg | 600 | mg | 200-400 | mg |
| Magnesium^{2,3} | 400 | mg | 400-800 | mg | 400-800 | mg | 400-800 | mg | 400-800 | mg | 400-800 | mg |

¹Alternative to ubiquinol.
²Optional.
³Constipation contraindicated.

TABLE 16
Microcontaminant and Free Radical Removal

| | | | Stage 1-4 | |
|---|---|---|---|---|
| Microcontaminant/Free Radical | Maintenance | | Dose/ | |
| Treatment | Dose | | Leaky Gut | |
| Modified Citrus Pectin¹ | 5-10 | g | 10-20 | g |
| Oral Chelation and Antioxidant Detox | | | | |
| Formula^{1,2} | | | | |
| Calcium Disodium EDTA | 500 | mg | 500 | mg |
| N-Acetyl Cysteine | 500 | mg | 500 | mg |
| Chlorella | 375 | mg | 375 | mg |
| Rosemary Leaf Extract | 275 | mg | 275 | mg |
| Turmeric Root Extract (95% curcuminoids) | 250 | mg | 250 | mg |
| Modified Citrus Pectin | 250 | mg | 250 | mg |
| Garlic Bulb | 250 | mg | 250 | mg |
| Broccoli Sprouts | 250 | mg | 250 | mg |
| Holy Basil Leaf Extract | 225 | mg | 225 | mg |
| Olive Leaf Extract | 166 | mg | 166 | mg |
| Silymarin | 150 | mg | 150 | mg |
| Calcium D-Glucarate | 125 | mg | 125 | mg |
| Green Tea Leaf Extract | 125 | mg | 125 | mg |
| Cilantro Leaf Extract | 125 | mg | 125 | mg |
| Wasabi Rhizome | 104 | mg | 104 | mg |
| Shilajit Mineral Resin Extract | 62 | mg | 62 | mg |
| R-Lipoic Acid | 50 | mg | 50 | mg |
| Plasma chelation therapy^{1,3} | | | | |
| Antioxidants⁴ | | | | |

¹Alternative toxin treatment, only one used at a time.
²Suggested use for Oral Chelation and Antioxidant Detox Formula: 1st cycle - ⅓ dose twice daily for 5 days then 9 days off; 2nd cycle - ⅔ dose twice daily for 5 days then 9 days off; 3rd cycle - full dose twice daily for 5 days then 30 days off. Same dose and cycle for all conditions being treated. Take on an empty stomach with at least 12 ounces of water.
³Administered by a skilled professional.
⁴Free radical treatment, preferably recycling antioxidant blend such as Rejuvenation Science ® Green C ™ or Advanced AntiOxidants, used in combination with any of the toxin treatments.

### Methods Include

One or more precipitating factors (such as those that harm the integrity of the mucosal milieu (in the gut, sinus, lungs, cardiovascular and lymph systems, and on the skin) and/or interfere with its intra- & extra-species signaling abilities), including, but not limited to, antibiotics, chlorine (oral, inhaled, and topical), alcohol (oral and topical), antibacterial cleaning products, acetaminophen, pesticides, fungicides, herbicides, hydrocarbons, phosphoric acid (soda), food contaminants (antibiotics, herbicides) and/or antibacterial or alcohol based mouthwash, are withheld from the patient undergoing treatment.

Irritating factors, such as fiber, roughage, fruit, raw vegetables, foods such as nuts, seeds, popcorn, all products containing corn, digestive enzymes, hydrochloric acid and/or allergens (oral), are temporarily removed/reduced, such as until the mucosal milieu is restored.

Pharmaceutical drugs for symptomatic relief can be administered to the patient, with the exception of immune destroying drugs, such as Infliximab, wide spectrum antibiotics, and long-term antibiotic use. Effectiveness of the treatment may be decreased if used in conjunction with long-term use of other drugs where the known side effects include intestinal distress, dry membranes, mitochondrial damage and other symptoms of mucosal milieu dysbiosis.

### Instructions for Use

### Primary Therapy

Begin therapy with a maintenance dose of the composition or plurality of compositions, and titrate up to the full dose as determined based on the diagnosis in the patient. The interval between dose increases may be daily or longer, such as one day, two days, three days, or more. Maintain recommended treatment dose until symptoms are alleviated and stable health is achieved. Then taper down any pharmaceutical drugs that are being administered along with the treatment regimen, which may be masking symptoms. Symptom flares while tapering pharmaceutical drugs may occur. At any sign of reoccurring symptoms (flare), stop tapering or titrate back up the pharmaceutical drugs until the symptoms in the patient are brought under control. Continue slowly tapering any pharmaceutical drugs until they are no longer being administered or the minimum dose for managing symptoms has been reached.

When the patient is stable and either no longer taking pharmaceutical drugs or taking the minimum dose of pharmaceutical drugs required to manage symptoms, taper the symptomatic relief component dose to zero, followed by tapering the cellular bioenergetics component to a maintenance dose. Next, taper the organic constituents of tissue component to a maintenance dose and concurrently slowly taper the mucosal milieu treatment component until a maintenance dose is achieved.

### Dosing

All components are best taken as a divided dose. All micronutrients are best taken with food. The mucosal milieu formula is best taken 1 hour before or after a meal with room temperature or colder liquid. Water is suggested.

If the patient was not breastfed for at least one year, titrate treatment level up to the high end of the range.

For treatment of diseases of the oral cavity, the oral dose of the mucosal milieu formula should not be encapsulated, but swished around in the mouth for a minute, then swallowed. Do not eat or drink for 10-20 minutes afterward.

Children's doses should be determined based on the ratio of child to adult body weight.

If a dose is missed, it may be taken at any time up to and including the time for the next dose.

### Advanced Therapy

Toxin removal could cause flu-like symptoms and/or exacerbate the primary condition. Begin toxin removal after stability is achieved with other treatment components.

Polyp removal will take a long time. Do not start this part of the protocol until stability is achieved with the other components. Expect polyp treatment to take 6-24 months or longer.

### Treatment Complexities

The treatment regimen manages many vectors at once working within a system with multiple areas of damage: restoring possibly multiple mucosal milieus to stop entry of contaminants and cascading immune responses, removing contaminants both in plasma and in tissue, assisting the adjacent or non-adjacent organ or system to heal, increasing mitochondrial energy production to promote healing, providing organ specific nutrients to enable healing, reducing symptoms to facilitate healing, moderating the immune system by removing immune triggers, and possibly tapering pharmaceutical drugs which may be masking further symptoms.

### Expectations

Expect to see initial response to treatment in a few days to a month. Time to return to a stable, healthy, normal condition may take up to 10% of the amount of time the patient has had the disease/problem.

Note: altering intestinal permeability by restoring the intestinal milieu may change (typically reduce) absorption of any pharmaceutical drugs and thus alter their effectiveness, while likely also reducing the need for the drugs. Similar reactions may be noted for drugs delivered as a nasal mist, topically, or intravenously.

### Safety and Warnings

All of the components have a long history of safe use by themselves. Many of them are individually determined to be GRAS. There is no expected harm to use even higher doses of the mucosal milieu components for potentially faster results. Each of these components has been used in significantly higher doses with no reported side effects.

Since patients who require this therapy generally have immune system dysbiosis, there is the chance of an allergic reaction to any component. If there are any known allergies to individual components, the patient should skip that component, with the exception of high quality colostrum, which should contain almost no milk proteins. For a person allergic to milk proteins, they may use beef protein powder as an alternate to whey protein.

One of the “side effects” of mucosal milieu therapy is that allergies and sensitivities will decrease or disappear, as well as any non-targeted “auto-immune disease.”

A known side effect to probiotics occurs if the patient has an abundance of harmful bacteria in their system. The probiotics will try to overwhelm and destroy the harmful bacteria, and this “fight” may cause temporary indigestion, nausea, and diarrhea, potentially similar to food poisoning. This is actually a good sign, but it is why we recommend starting the dose low (at the maintenance level) and tapering up as quickly as tolerated.

### Monitoring Progress

To monitor progress, in addition to noting and monitoring symptomology using specific disease scoring, the protocol recommends screenings including baseline (before treatment) plasma hsCRP, 25(OH)D, photos of visible organ dysbiosis, and notation of any other “unrelated” conditions that the patient presents with, along with details on the “unrelated” conditions/symptoms.

### Maintenance Therapy

Ongoing treatment with a mucosal milieu maintenance dose is required once the patient is cured and doses are tapered down to maintenance dose level. Maintenance is required for as long as the patient is exposed to primary antibiotics, antibiotic residue in food and water, processed food, herbicide residue in standard or GM food, chlorine in municipal tap water, cleaning chemicals or sanitizers, high sugar and carb consumption, alcohol, hydrocarbon emissions from cars, power plants, refineries, etc., or a combination of these contaminants that damage the tissue barrier.

### Example 3. Protocol for Treatment of Cardiovascular Dysfunction

### Diagnosis

The following table categorizes disease diagnosis and protocol dosing from lowest diagnosable manifestation to most severe. For difficult cases, the dosing can be doubled. To save costs on maintenance, dosing may be halved, but that increases risk that the condition will reappear.

TABLE 17
Manifestation stages of cardiovascular conditions
associated with tissue barrier dysfunction.

| | Stage 1 | Stage 2 | Stage 3 | Stage 4 |
|---|---|---|---|---|
| Mitochondrial | high blood | stable | congestive | severe CHF, |
| dysfunction | pressure, | angina, | heart | dilated |
| | mitral valve | arrhythmia | failure | cardio- |
| | prolapse | | (CHF) | myopathy |
| Arterial | high blood | arterio- | | |
| dysfunction | pressure | sclerosis | | |

### Specific Treatments

Patients diagnosed with cardiovascular dysfunction are administered a treatment regimen that includes a composition or plurality of compositions comprising one or more of the items listed in Tables 18-23 (provided as adult daily dosage, which is best taken as a divided dose, and adjusted for weight, such as for children).

TABLE 18
Mucosal milieu treatment - oral

| | Maintenance | | Stage 1 | | Stage 2 | | Stage 3 | | Stage 4 | |
|---|---|---|---|---|---|---|---|---|---|---|
| Ingredient | Dose | | Dose | | Dose | | Dose | | Dose | |
| Probiotic blend^{1,2} | 30-60 | B³ | 60-120 | B | 120-240 | B | 180-360 | B | 180-720 | B |
| Colostrum⁴ | 1-2 | g | 2-4 | g | 4-8 | g | 6-12 | g | 6-24 | g |
| Prebiotics | 250-500 | mg | 0.5-1 | g | 1-2 | g | 1.5-3 | g | 1.5-6 | g |
| Whey Protein | 5-10 | g | 10-20 | g | 20-40 | g | 30-60 | g | 30-120 | g |
| Beef Protein⁵ | 5-10 | g | 10-20 | g | 20-40 | g | 30-60 | g | 30-120 | g |
| Bacillus coagulans⁶ | 5 | B | 10 | B | 20 | B | 20 | B | 20 | B |

¹A complex blend of acid-resistant probiotic strains. Refrigeration is suggested to maintain potency.
²Our formulation is loaded with >4 times the label claim of CFU (live colony forming units) (i.e.: 110 billion CFU at time of bottling is guaranteed to deliver 25 billion CFU at expiration date when stored under refrigeration). So, the actual CFU at time of use for the product labeled 25 billion CFU may vary from 100 billion to 25 billion. Stability testing on this product indicates the formula drops to 25 billion 14 months after bottling when stored at ambient room temperature (70 degrees F.).
³B = billion CFU (Colony Forming Units) (live organisms)
⁴High in immunoglobulin content, high in protein-rich polypeptides (PRP), low in milk proteins.
⁵Alternative to Whey Protein for patients who are allergic/sensitive to milk/casein products.
⁶For use in conjunction with probiotic blend when environmental factors make the preferred probiotic blend less effective: in conjunction with antibiotic therapy or when traveling and refrigeration not available.

TABLE 19
Mucosal Milieu Formula (spray mist)¹

| Ingredient | Dose | |
|---|---|---|
| Probiotic blend² | 60 | B |
| Colostrum | 100 | mg |
| Prebiotics | 100 | mg |
| Whey Protein | 100 | mg |
| Beef Protein³ | 100 | mg |

¹Used if patient presents with comorbid conditions such as allergy, sinusitis, any form of COPD, or history of smoking. In a base of spring water or vegetable glycerin delivered as a spray mist or delivered by a form of inhalation therapy, such as using a vaporizer using the lowest temperature setting. Inhalation treatment will be delivered 1-2x daily for maintenance, 2-4x daily for stage 1, 4-8x daily for stage 2, and 6-12x daily for stages 3 and 4.
²Same mix of probiotic strains as oral formula.
³Alternative to Whey Protein for patients who are allergic/sensitive to milk/casein products.

TABLE 20
Organic Constituents of Tissue - oral

| | Maintenance | | Stage 1 | | Stage 2 | | Stage 3 | | Stage 4 | |
|---|---|---|---|---|---|---|---|---|---|---|
| Ingredient | Dose | | Dose | | Dose | | Dose | | Dose | |
| Krill oil¹ | 0.5 | g | 0.5-1 | g | 1-2 | g | 2-4 | g | 4-8 | g |
| Fish oil² | 3 | g | 3-5 | g | 5-10 | g | 10-20 | g | 20-40 | g |
| Phospholipids^{2,3} | 150 | mg | 150-300 | mg | 300-60 | mg | 600-1200 | mg | 1.2-2.4 | g |
| Micronutrient Therapy⁴ | | | | | | | | | | |

| | |
|---|---|
| Vitamin D3 | typically 5,000 to |
| | 10,000 IU/day⁵ |

¹Containing EPA, DHA, and other PUFAs (polyunsaturated fatty acids) bound to 2 or more of 69 different phospholipid molecules, plus the antioxidant astaxanthin.
²Alternative to krill oil
³As phospholipid complex consisting of Phosphatidyl Serine, Phosphatidyl Choline, Phosphatidyl Ethanolamine, Phosphatidyl Inositol, Phosphatidic Acid, and Acetylated Phosphatidyl Ethanolamine.
⁴Sophisticated multivitamin such as Rejuvenation Science Maximum Vitality taken at standard dose with food.
⁵Dosed to achieve 80-100 ng/ml 25(OH)D plasma level.

TABLE 21
Symptomatic Relief - Vascular

| | | | |
|---|---|---|---|
| | Maintenance | Stage 1 | Stage 2 |
| Ingredient | Dose | Dose | Dose |

| | | | | |
|---|---|---|---|---|
| Fibrin optimizer | 2000 | FU¹ | 4000-6000 | FU |
| Hawthorne Berry | 200 | mg | 400 | mg |
| Ginkgo Biloba | 120 | mg | 240 | mg |

¹Fibrinolytic Units

TABLE 22
Cellular Bioenergetics - oral

| | Maintenance | | Stage 1 | | Stage 2 | | Stage 3 | | Stage 4 | |
|---|---|---|---|---|---|---|---|---|---|---|
| Ingredient | Dose | | Dose | | Dose | | Dose | | Dose | |
| CoQ10 (ubiquinol) | 50 | mg | 100 | mg | 100-200 | mg | 200-400 | mg | 300-600 | mg |
| CoQ10 (ubiquinone)¹ | 400-500 | mg | 0.8-1 | g | 0.8-2 | g | 1.6-4 | g | 2.4-6 | g |
| L-Carnitine | 500 | mg | 0.5-1 | g | 1-2 | g | 2-2.5 | g | 2.5-3.5 | g |
| PQQ | — | | 10 | mg | 20 | mg | 30 | mg | 40 | mg |
| D-ribose | 5 | g | 5-10 | g | 10-15 | g | 10-15 | g | 15 | g |
| Alpha-Lipoic Acid | 50 | mg | 100 | mg | 200 | mg | 400 | mg | 600 | mg |
| Magnesium² | 400 | mg | 400-800 | mg | 400-800 | mg | 400-800 | mg | 400-800 | mg |

¹Alternative to ubiquinol.
²Constipation contraindicated.

TABLE 23
Microcontaminant and Free Radical Removal

| Microcontaminant/Free Radical | Maintenance | | Stage 1-4 | |
|---|---|---|---|---|
| Treatment | Dose | | Dose | |
| Modified Citrus Pectin¹ | 5-10 | g | 10-20 | g |
| Oral Chelation and Antioxidant Detox | | | | |
| Formula^{1,2} | | | | |
| Calcium Disodium EDTA | 500 | mg | 500 | mg |
| N-Acetyl Cysteine | 500 | mg | 500 | mg |
| Chlorella | 375 | mg | 375 | mg |
| Rosemary Leaf Extract | 275 | mg | 275 | mg |
| Turmeric Root Extract (95% curcuminoids) | 250 | mg | 250 | mg |
| Modified Citrus Pectin | 250 | mg | 250 | mg |
| Garlic Bulb | 250 | mg | 250 | mg |
| Broccoli Sprouts | 250 | mg | 250 | mg |
| Holy Basil Leaf Extract | 225 | mg | 225 | mg |
| Olive Leaf Extract | 166 | mg | 166 | mg |
| Silymarin | 150 | mg | 150 | mg |
| Calcium D-Glucarate | 125 | mg | 125 | mg |
| Green Tea Leaf Extract | 125 | mg | 125 | mg |
| Cilantro Leaf Extract | 125 | mg | 125 | mg |
| Wasabi Rhizome | 104 | mg | 104 | mg |
| Shilajit Mineral Resin Extract | 62 | mg | 62 | mg |
| R-Lipoic Acid | 50 | mg | 50 | mg |
| Plasma chelation therapy^{1,3} | | | | |
| Antioxidants⁴ | | | | |

¹Alternative microcontaminant treatment, only one used at a time.
²Suggested use for Oral Chelation and Antioxidant Detox Formula: 1st cycle - ⅓ dose twice daily for 5 days then 9 days off; 2nd cycle - ⅔ dose twice daily for 5 days then 9 days off; 3rd cycle - full dose twice daily for 5 days then 30 days off. Same dose and cycle for all conditions being treated. Take on an empty stomach with at least 12 ounces of water.
³Administered by a skilled professional.
⁴Free radical treatment, preferably recycling antioxidant blend such as Rejuvenation Science ® Green C ™ or Advanced AntiOxidants.

### Methods Include

One or more precipitating factors (such as those that harm the integrity of the mucosal milieu (in the gut, sinus, lungs, cardiovascular and lymph systems, and on the skin) and/or interfere with its intra- & extra-species signaling abilities), including, but not limited to, antibiotics, chlorine (oral, inhaled, and topical), alcohol (oral and topical), antibacterial cleaning products, acetaminophen, pesticides, fungicides, herbicides, hydrocarbons, phosphoric acid (soda), food contaminants (antibiotics, herbicides) and/or antibacterial or alcohol based mouthwash, are withheld from the patient undergoing treatment.

Irritating factors, such as excess homocysteine and/or allergens (oral), are temporarily removed/reduced, such as until the mucosal milieu is restored.

Pharmaceutical drugs for symptomatic relief can be administered to the patient, with the exception of statins, beta blockers, wide spectrum antibiotics and long-term antibiotic use. Effectiveness of the treatment may be decreased if used in conjunction with long-term use of other drugs where the known side effects include mitochondrial damage.

### Treatment Strategy

Recognize the chemicals that damage the human tissue barrier and stop exposure to those tissue barrier toxins.

Restore the tissue barrier to enable restoration of membrane integrity (human tissue barrier and physical cellular junctions) to prevent contaminants from entering the body (primarily GI and sinus/lungs), while allowing nutrients to enter and waste materials to exit.

Restore the glycocalyx (vascular milieu) to strengthen the vascular barrier to minimize contaminants entering cardiovascular tissue and mitochondria, and enhance excretion of contaminants, while allowing nutrients to enter and waste materials to exit. Restoring the glycocalyx will also minimize need for protective plaques to form, facilitating healthy blood flow and blood pressure.

Enhance cellular bioenergetics to facilitate growth and healing of diseased tissue/organs in GI, lungs, heart, and kidneys.

Restore and enhance human tissue barrier in the skin (derma milieu) to support natural vitamin D production.

Provide micronutrient support, lipid replacement, and other organic constituents of tissue for healthy growth and healing.

Continue symptom treatment with current and alternative/adjunctive therapies. Decrease symptomatic treatment of the immune response to contaminants as the natural immune response abates due to restoration of the human tissue barrier and membrane barriers.

Chelate contaminants that have already entered the body and the cardiovascular system. Removing contaminants is a key step to ongoing healthy function.

Decrease treatments to maintenance level to maintain integrity of the tissue barrier and tissue, as ongoing exposure to some contaminants is unavoidable.

### Instructions for Use

### Primary Therapy

Discontinue exposure to primary sources of precipitating factors and as many secondary sources as possible. Primary sources include antibiotics, chlorine in drinking water, chlorine mist in shower or bath, alcohol (oral and topical). Secondary sources include antibacterial cleaning products, acetaminophen, pesticides, fungicides, herbicides, hydrocarbons, phosphoric acid (soda), food contaminants (antibiotics, herbicides, pesticides) especially in GMO foods. A chlorine drinking water filter and a chlorine shower filter are recommended.

Re-exposure to primary precipitating factors may require restarting therapy.

If a course of oral antibiotics is absolutely necessary, double the dose of the oral mucosal milieu formula during antibiotic therapy and for two weeks after, including the optional Bacillus Coagulans doses.

Begin mucosal milieu (oral and inhalation), organic constituents, symptomatic relief, and cellular bioenergetics therapies with maintenance dose and quickly titrate up to full dose based on diagnosis. May increase dose every day or two. Maintain recommended treatment dose until symptoms are gone and healthy stability established for at least 60 days.

Microcontaminant removal could cause flu-like symptoms and/or temporarily exacerbate the primary condition. Begin microcontaminant removal after 60 days treatment with other components. Continue micronutrient removal for 2-12 months, or for 60 days after symptoms have abated. Microcontaminant removal should be done slowly—increasing the dose is not recommended. Microcontaminant removal is required to obtain best results and to minimize risk of relapse.

Taper down on pharmaceutical drugs, which may be masking symptoms, so don't be surprised with flares while tapering pharmaceutical drugs. At any sign of recurring symptoms (flare), stop tapering or titrate back up pharma drugs until patient is stable. Continue slowly tapering pharmaceutical drugs.

When patient is stable and no longer taking pharmaceutical drugs, taper Symptomatic Relief to 0, then taper Cellular Bioenergetics to maintenance dose, then taper Organic Constituents of Tissue to maintenance dose and concurrently slowly taper mucosal milieu therapies until maintenance dose is achieved.

### Dosing

All components are best taken as a divided dose. All micronutrients are best taken with food. The mucosal milieu formula is best taken 1 hour before or after a meal with room temperature or colder liquid. Filtered or spring water is suggested.

If patient was not breastfed for at least one year, titrate treatment level up to high end of the range.

Children's doses should be determined based on the ratio of child to adult body weight.

For difficult cases, the dosing can be doubled. To save costs on maintenance, dosing may be halved, but that increases risk that the condition will reappear in patients who have already demonstrated susceptibility.

If a dose is missed, it may be taken at any time up to and including the time for the next dose.

### Advanced Therapy

### Treatment Complexities

The treatment regimen manages many vectors at once working within a system with multiple areas of damage: restoring possibly multiple mucosal milieus to stop entry of contaminants and cascading immune responses, removing contaminants both in plasma and in tissue, assisting the adjacent or non-adjacent organ or system to heal, increasing mitochondrial energy production to promote healing, providing organ specific nutrients to enable healing, reducing symptoms to facilitate healing, moderating the immune system by removing immune triggers, and possibly tapering pharmaceutical drugs which may be masking further symptoms.

### Expectations

Expect to see initial response to treatment in a few days to a month. Time to return to a stable, healthy, normal condition may take up to 10% of the amount of time the patient has had the disease/problem.

Note: altering intestinal permeability by restoring the intestinal milieu may change (typically reduce) absorption of any pharmaceutical drugs and thus alter their effectiveness, while likely also reducing the need for the drugs. Similar reactions may be noted for drugs delivered as a nasal mist, topically, or intravenously.

### Safety and Warnings

All of the components have a long history of safe use by themselves. Many of them are individually determined to be GRAS. There is no expected harm to use even higher doses of the mucosal milieu components for potentially faster results. But faster initial results do not shorten the duration of the treatment. Each of these components has been used in significantly higher doses with no reported side effects.

Since patients who require this therapy generally have immune system dysbiosis, there is the chance of an allergic reaction to any component. If there are any known allergies to individual components, the patient should skip that component, with the exception of high quality colostrum, which should contain almost no milk proteins. For a person allergic to milk proteins, they may use beef protein powder as an alternate to whey protein.

One of the “side effects” of mucosal milieu therapy is that allergies and sensitivities will decrease or disappear, as well as any non-targeted “auto-immune disease.”

A known side effect to probiotics occurs if the patient has an abundance of harmful bacteria in their system. The probiotics will try to overwhelm and destroy the harmful bacteria, and this “fight” may cause temporary indigestion, nausea, and diarrhea, potentially similar to food poisoning. This is actually a good sign, but it is why we recommend starting the dose low (at the maintenance level) and tapering up as quickly as tolerated.

CoQ10-ubiquinol should not be suddenly discontinued due to concern of a sudden reduction of ATP production in heart patients noted in the literature.

### Monitoring Progress

To monitor progress, in addition to noting and monitoring symptomology using specific disease scoring, the protocol recommends screenings including baseline (before treatment) plasma hsCRP, homocysteine, 25(OH)D, and notation of any other “unrelated” conditions that the patient presents with, along with details on the “unrelated” conditions/symptoms.

### Maintenance Therapy

Ongoing mucosal milieu maintenance dose is required once the patient is cured and doses are tapered down to maintenance dose level. Maintenance is required so long as patient is exposed to primary antibiotics, antibiotic residue in food and water, processed food, herbicide residue in standard or GM food, chlorine in municipal tap water, cleaning chemicals or sanitizers, high sugar and carb consumption, alcohol, hydrocarbon emissions from cars, power plants, refineries, etc., or a combination of these contaminants that damage the tissue barrier primarily in the gut, lungs or vascular system.

### EXEMPLARY EMBODIMENTS

The invention is further described by the following embodiments. The features of each of the embodiments are combinable with any of the other embodiments where appropriate and practical.

### Embodiment 1

In one embodiment, there is provided a composition comprising: 1) a probiotic component, 2) colostrum, 3) a protein component and 4) a detoxification component.

### Embodiment 2

The composition of embodiment 1 further comprising one or more components selected from the group consisting of 5) a prebiotic component, 6) a tissue constituent component, 7) a symptomatic relief component, 8) a cellular bioenergetics component, 9) a tissue healing component, and 10) an enzyme component.

### Embodiment 3

The composition of embodiment 1 or 2, wherein the probiotic component comprises: 1) at least one microorganism of the Bacillus genus; 2) at least one microorganism of the Lactobacillus genus; 3) at least one microorganism of the Bifidobacterium genus, 4) at least one microorganism of the Escherichia genus, 5) at least one microorganism of the Streptococcus genus and 6) at least one microorganism of the Lactococcus genus.

### Embodiment 4

The composition of embodiment 3, wherein the microorganism of the Bacillus genus is one or more of: Bacillus coagulans and Bacillus subtilis.

### Embodiment 5

The composition of embodiment 3 or 4, wherein the microorganism of the Lactobacillus genus is one or more of: Lactobacillus paracaei, Lactobacillus casei, Lactobacillus bulgaricus, Lactobacillus acidophilus, Lactobacillus rhamnosis, Lactobacillus rhamnosis GR-1®, Lactobacillus plantarum, Lactobacillus johnsonii, Lactobacillus salivarius, Lactobacillus fermentum, Lactobacillus helveticus, Lactobacillus gasseri, Lactobacillus reuteri, Lactobacillus reuteri Protectis, Lactobacillus reuteri Prodentis and Lactobacillus reuteri RC-14®.

### Embodiment 6

The composition of any one of embodiments 3-5, wherein the microorganism of the Bifidobacterium genus is one or more of: Bifidobacterium lactis, Bifidobacterium breve, Bifidobacterium longum, Bifidobacterium infantis, Bifidobacterium animalis subsp. lactis, and Bifidobacterium bifidum.

### Embodiment 7

The composition of any one of embodiments 3-6, wherein the microorganism of the Escherichia genus is E. coli.

### Embodiment 8

The composition of any one of embodiments 3-7, wherein the microorganism of the Streptococcus genus is Streptococcus thermophiles.

### Embodiment 9

The composition of any one of embodiments 3-8, wherein the microorganism of the Lactococcus genus is one or more of: Lactococcus thermophiles and Lactococcus lactis.

### Embodiment 10

The composition of any one of embodiments 3-9, wherein the probiotic component further comprises one or more of: L. cellobiosus, L. leichmannii, and L. salivaroes.

### Embodiment 11

The composition of embodiment 1 or 2, wherein the probiotic component comprises all the microorganisms of Table 1.

### Embodiment 12

The composition of embodiment 1 or 2, wherein the probiotic component comprises all the microorganisms of Table 2.

### Embodiment 13

The composition of any one of embodiments 1-12, wherein the colostrum is substantially non-allergenic.

### Embodiment 14

The composition of any one of embodiments 1-13, wherein the colostrum is from human, cow, sheep or goat.

### Embodiment 15

The composition of any one of embodiments 2-14, wherein the prebiotic component comprises one or more of: inulin, galactooligosaccharide (GOS), fructooligosaccharide (FOS), xylooligosaccharide (XOS), mannan-oligosaccharide (MOS) and polydextrose.

### Embodiment 16

The composition of embodiment 15, wherein the prebiotic component comprises fructooligosaccharide.

### Embodiment 17

The composition of any one of embodiments 1-16, wherein the probiotic component, colostrum, and protein component are provided in one of the amounts listed in Table 3.

### Embodiment 18

The composition of any one of embodiments 1-17, wherein the detoxification component comprises modified citrus pectin or one or more of the items listed in Table 4 for oral chelation and antioxidant detox formula in one of the amounts listed.

### Embodiment 19

The composition of any one of embodiments 2-18, wherein the prebiotic component is provided in one of the amounts listed in Table 3.

### Embodiment 20

The composition of any one of embodiments 2-19, wherein the tissue constituent component comprises one or more of the items listed in Table 5.

### Embodiment 21

The composition of embodiment 20, wherein the tissue constituent component comprises one or more of the items listed in Table 5 in one of the amounts listed.

### Embodiment 22

The composition of any one of embodiments 2-21, wherein the tissue constituent component comprises the organic constituent of the GI tissue.

### Embodiment 23

The composition of any one of embodiments 2-22, wherein the symptomatic relief component comprises one or more of the items listed in Table 6.

### Embodiment 24

The composition of embodiment 23, wherein the symptomatic relief component comprises one or more of the items listed in Table 6 in one of the amounts listed.

### Embodiment 25

The composition of any one of embodiments 2-24, wherein the cellular bioenergetics component comprises one or more of the items listed in Table 7.

### Embodiment 26

The composition of embodiment 25, wherein the cellular bioenergetics component comprises one or more of the items listed in Table 7 in one of the amounts listed.

### Embodiment 27

The composition of any one of embodiments 2-26, wherein the tissue healing component comprises one or more of the items listed in Table 8.

### Embodiment 28

The composition of embodiment 27, wherein the tissue healing component comprises one or more of the items listed in Table 8 in one of the amounts listed.

### Embodiment 29

The composition of any one of embodiments 2-28, wherein the enzyme component comprises one or more of the items listed in Table 9.

### Embodiment 30

The composition of embodiment 29, wherein the enzyme component comprises one or more of the items listed in Table 9 in one of the amounts listed.

### Embodiment 31

In one embodiment, there is provided a method of enhancing, protecting or repairing the integrity of the tissue barrier adjacent to or on a tissue of an individual, comprising administering to the individual an effective amount of the composition of any one of embodiments 1-30.

### Embodiment 32

In one embodiment, there is provided a method of preventing or treating inflammation of a tissue of an individual, comprising administering to the individual an effective amount of the composition of any one of embodiments 1-30.

### Embodiment 33

In one embodiment, there is provided a method of preventing or repairing damage in an individual, comprising administering to the individual an effective amount of the composition of any one of embodiments 1-30.

### Embodiment 34

In one embodiment, there is provided a method of mitigating leakage of a tissue barrier and/or a mucosal membrane adjacent to or on a tissue of an individual, comprising administering to the individual an effective amount of the composition of any one of embodiments 1-30.

### Embodiment 35

In one embodiment, there is provided a method of reducing the risk of a disease or condition in an individual, comprising administering to the individual an effective amount of the composition of any one of embodiments 1-30.

### Embodiment 36

In one embodiment, there is provided a method of reducing a side effect associated with the administration of an antibiotic in an individual, comprising administering to the individual an effective amount of the composition of any one of embodiments 1-30.

### Embodiment 37

In one embodiment, there is provided a method of reducing an adverse effect in an individual associated with a) exposure of the individual to an environmental contaminant or non-natural substance; or b) abnormal levels of a naturally occurring substance in the individual, comprising administering to the individual an effective amount of the composition of any one of embodiments 1-30.

### Embodiment 38

In one embodiment, there is provided a method of preventing or treating a disease or condition in an individual, comprising administering to the individual an effective amount of the composition of any one of embodiments 1-30.

### Embodiment 39

In one embodiment, there is provided a method of preventing or treating a disease or condition in an individual, comprising administering to the individual a plurality of compositions comprising 1) a probiotic component, 2) colostrum, 3) a protein component, and 4) a detoxification component.

### Embodiment 40

The method of embodiment 39, wherein the plurality of compositions further comprises one or more of 5) a prebiotic component, 6) a tissue constituent component, 7) a symptomatic relief component, 8) a cellular bioenergetics component, 9) a tissue healing component, and 10) an enzyme component.

### Embodiment 41

The method of any one of embodiments 38-40, wherein the disease or condition is associated with a disturbance in the integrity of one or more tissue barriers associated with one or more tissue in the individual.

### Embodiment 42

The method of any one of embodiments 38-41, wherein the disease or condition is selected from the group consisting of GI conditions, respiratory conditions, skin conditions, and autoimmune diseases.

### Embodiment 43

The method of any one of embodiments 38-42, further comprising determining a manifestation stage of the disease or condition and selecting the composition or plurality of compositions according to the affected tissues and manifestation stage.

### Embodiment 44

In one embodiment, there is provided a method of simultaneously treating comorbid conditions in an individual, comprising administering to the individual an effective amount of the composition of any one of embodiments 1-30.

### Embodiment 45

In one embodiment, there is provided a method of simultaneously enhancing, protecting or repairing the integrity of multiple tissue barriers located in different regions in the body of an individual, comprising administering to the individual an effective amount of the composition of any one of embodiments 1-30.

### Embodiment 46

In one embodiment, there is provided a method of enhancing, protecting or repairing the integrity of the blood-brain barrier in an individual, comprising administering to the individual an effective amount of the composition of any one of embodiments 1-30.

### Embodiment 47

In one embodiment, there is provided a method of treating diseases associated with brain or neurological dysfunction in an individual, comprising administering to the individual an effective amount of the composition of any one of embodiments 1-30.

### Embodiment 48

In one embodiment, there is provided a method of reducing the risk of one or more diseases associated with inflammation in an individual, comprising administering to the individual an effective amount of the composition of any one of embodiments 1-30.

### Embodiment 49

The method of embodiment 48, wherein the diseases are selected from the group consisting of cardiovascular disease, food allergies, and sinus allergies.

### Embodiment 50

In one embodiment, there is provided a method of treating diseases associated with exposure to one or more contaminants in an individual, comprising administering to the individual an effective amount of the composition of any one of embodiments 1-30.

### Embodiment 51

The method of embodiment 50, wherein the contaminants are selected from the group consisting of chlorine, herbicides, pesticides, antibiotics in foods and drinking or bathing water, hydrocarbons, tar, nicotine, smoke, and heavy metals.

### Embodiment 52

In one embodiment, there is provided a method of protecting a tissue after surgery and promoting post-surgical healing of the tissue in an individual, comprising administering to the individual an effective amount of the composition of any one of embodiments 1-30.

### Embodiment 53

The method of embodiment 52, wherein the tissue is GI tissue and the surgery is GI surgery.

### Embodiment 54

The method of any one of embodiments 31-53, wherein the individual is an infant, a child, an adult or a pregnant female.

### Embodiment 55

The method of embodiment 54, wherein the individual is human.

Although the foregoing invention has been described in some detail by way of illustration and example for purposes of clarity of understanding, it will be apparent to those skilled in the art that certain changes and modifications may be practiced without departing from the invention. Therefore, the descriptions and examples should not be construed as limiting the scope of the invention.

All patents, patent applications, documents, and articles cited herein are incorporated by reference in their entireties.

1. A composition comprising: 1) a probiotic component, 2) colostrum, 3) a protein component and 4) a detoxification component.

2. The composition of claim 1, further comprising one or more components selected from the group consisting of a prebiotic component, a tissue constituent component, a symptomatic relief component, a cellular bioenergetics component, a tissue healing component, and an enzyme component.

3. The composition of claim 1, wherein the probiotic component comprises: 1) at least one microorganism of the Bacillus genus; 2) at least one microorganism of the Lactobacillus genus; 3) at least one microorganism of the Bifidobacterium genus; 4) at least one microorganism of the Escherichia genus; 5) at least one microorganism of the Streptococcus genus; and 6) at least one microorganism of the Lactococcus genus.

4. The composition of claim 3, wherein the microorganism of the Bacillus genus is one or more of: Bacillus coagulans and Bacillus subtilis.

5. The composition of claim 3, wherein the microorganism of the Lactobacillus genus is one or more of: Lactobacillus paracaei, Lactobacillus casei, Lactobacillus bulgaricus, Lactobacillus acidophilus, Lactobacillus rhamnosis, Lactobacillus rhamnosis GR-1®, Lactobacillus plantarum, Lactobacillus johnsonii, Lactobacillus salivarius, Lactobacillus fermentum, Lactobacillus helveticus, Lactobacillus gasseri, Lactobacillus reuteri, Lactobacillus reuteri Protectis, Lactobacillus reuteri Prodentis and Lactobacillus reuteri RC-14®.

6. The composition of claim 3, wherein the microorganism of the Bifidobacterium genus is one or more of: Bifidobacterium lactis, Bifidobacterium breve, Bifidobacterium longum, Bifidobacterium infantis, Bifidobacterium animalis subsp. lactis, and Bifidobacterium bifidum.

7. The composition of claim 3, wherein the microorganism of the Escherichia genus is E. coli.

8. The composition of claim 3, wherein the microorganism of the Streptococcus genus is Streptococcus thermophiles.

9. The composition of claim 3, wherein the microorganism of the Lactococcus genus is one or more of: Lactococcus thermophiles and Lactococcus lactis.

10. The composition of claim 3, wherein the probiotic component further comprises one or more of: L. cellobiosus, L. leichmannii, and L. salivaroes.

11-21. (canceled)

22. The composition of claim 2, wherein the tissue constituent component comprises an organic constituent of a GI tissue.

23-30. (canceled)

31. A method of enhancing, protecting or repairing the integrity of the tissue barrier adjacent to or on a tissue of an individual, comprising administering to the individual an effective amount of the composition of claim 1.

32. A method of preventing or treating inflammation of a tissue of an individual, comprising administering to the individual an effective amount of the composition of claim 1.

33-53. (canceled)

54. The method of claim 31, wherein the individual is an infant, a child, an adult or a pregnant female.

55. The method of claim 54, wherein the individual is human.
